(19) United States
(12) Patent Application Publication
(10) Pub. No.: US 2007/0213586 A1
(43) Pub. Date: Sep. 13, 2007

(54) Endoscope system and adapter applied to this endoscope system

(75) Inventors: Kenji Hirose, Tokyo (JP); Koji Yasunaga, Tokyo (JP); Toshiya Sugai, Tokyo (JP)

Correspondence Address: Thomas Spinelli;Scully, Scott, Murphy & Presser, Suite 300, 400 Garden City Plaza, Garden City, NY, 11530, US

(73) Assignee: Olympus Medical Systems Corp., Tokyo (JP)

(21) Appl. No.: 11/713,273

(22) Filed: Mar. 2, 2007

(30) Foreign Application Priority Data
Mar. 7, 2006 (JP) 2006-061751

(51) Int. Cl. A61B 1/04; A61B 1/06

(52) U.S. Cl. 600/112; 600/160; 600/109; 600/178

(57) ABSTRACT

This endoscope system comprises image pickup means having an image pickup device, illumination light ejecting means having an illumination light ejection portion, an endoscope having an insertion portion and having an illumination light incident portion and an optical image ejection portion for ejecting subject light, a first connection portion for attaching the endoscope to the image pickup means so that the subject light can be captured, a second connection portion for attaching the endoscope to the illumination light ejecting means so that the light from the illumination light ejection portion can enter the illumination light incident portion, an optical path having a first adapter connection portion attachable to the first connection portion and a second adapter connection portion attachable to the second connection portion for capturing the subject image, and an adapter having an illumination optical system for guiding the illumination light to the image pickup region.

This application claims the benefit of Japanese Application No. 2006-061751 filed Mar. 7, 2006, the contents of which are incorporated by this reference.

### BACKGROUND OF THE INVENTION

1. Field of the Invention

The present invention relates to an endoscope system and more particularly to an endoscope system configured so that an endoscope and an image pickup device are detachable and used in surgeries, particularly in neurosurgery, otolaryngology, orthopedic/plastic surgery, obstetrics and gynecology and the like.

2. Description of the Related Art

In response to a demand for low-invasive operations, surgeries using an endoscope system have been widely performed.

A conventional endoscope system used in surgeries in general comprises an endoscope to be inserted into a body cavity, an image pickup apparatus such as a so-called television camera (hereinafter referred to as TV camera) or the like having a camera adapter configured capable of being connected to the endoscope and including an image forming lens and the like and a camera head configured capable of being connected to this camera adapter and including an image pickup device for capturing an observed image formed by the endoscope and the like, a light guide connected to the endoscope for guiding an illumination luminous flux from a light source device, and a fixing device such as a support arm and the like for fixing these devices to an operated portion. In this case, the camera head and the adapter constituting the image pickup apparatus may be configured integrally.

Also, at a surgery using an endoscope, a procedure such as incision in the vicinity of an operated portion may be performed before insertion of the endoscope. In this case, since the range of the incision is small, an operator needs to perform the procedure by looking into the procedure portion.

Also, since the procedure is performed by the operator, who is looking into the procedure portion as mentioned above, a plurality of staff can not observe the portion concerned.

Then, in order to solve the problem, there can be an idea that a microscope for operation is used for observing the procedure portion. However, a high magnification of the microscope for operation is not necessary for observation of the procedure portion, and that is not efficient in terms of economy as well as an installation space. Moreover, in this case, it is necessary to replace the microscope used for the procedure before the operation by an endoscope for the operation after the procedure, which takes labor and time for the operation and is not considered to be an efficient solution.

Then, an endoscope system was proposed that, at a procedure such as an incision before a surgery using an endoscope, an image pickup apparatus in an endoscope system is used as if it is a microscope as TV camera for observing an operated portion so as to capture an image of the incised portion so that an incision procedure or the like before the operation can be performed while observing a monitor screen in Japanese Unexamined Patent Application Publication No. 2005-645, for example.

The endoscope system disclosed in the above Japanese Unexamined Patent Application Publication No. 2005-645 comprises an endoscope, a camera adapter configured detachable with respect to the endoscope, an image pickup apparatus configured detachable with respect to this camera adapter, a light guide connected to the endoscope, a support arm for supporting the camera adapter and fixing it at a desired arbitrary position and the like.

In this case, inside the camera adapter, optical system members such as an image rotation prism, a focus lens, a zoom lens and the like are incorporated, and it is configured to function as a TV camera for observing an operated portion by removing the endoscope from the camera adapter. And it is also configured so that an image of the operated portion is captured using the image pickup apparatus from which the endoscope has been removed and a pre-operation procedure can be performed while looking at the observation image.

According to this endoscope system, this can be used as an endoscope when the endoscope is attached to the image pickup apparatus, while it can also be used as a microscope as a TV camera for observing the operated portion when the endoscope is removed from the image pickup apparatus, and the above problem can be seemingly solved.

### SUMMARY OF THE INVENTION

An endoscope system according to the present invention comprises image pickup means having an image pickup device, illumination light ejecting means having an illumination light ejection portion, an endoscope having an insertion portion and having an illumination light incident portion for guiding illumination light to a base end of the insertion portion and an optical image ejection portion for ejecting light from a subject guided through the insertion portion, a first connection portion provided at the image pickup means for attaching the endoscope to the image pickup means so that the light from the subject ejected by the optical image ejection portion can be captured by the image pickup device, a second connection portion provided at the illumination light ejecting means for attaching the endoscope to the illumination light ejecting means so that the illumination light ejected by the illumination light ejection portion can enter the illumination light incident portion, an adapter having a first adapter connection portion which can be attached to the first connection portion instead of the endoscope and a second adapter connection portion which can be attached to the second connection portion instead of the endoscope, an optical path provided at the adapter for capturing an image of the subject by the image pickup device, and an illumination optical system provided at the adapter for guiding the illumination light ejected from the illumination light ejection portion into an image pickup region for image pickup through the optical path.

Advantages of the present invention will become more apparent from the following detailed description.

### BRIEF DESCRIPTION OF THE DRAWINGS

FIG. 1 is an outline configuration view showing an outline of an entire configuration when an endoscope system of a first embodiment of the present invention is used as an endoscope;

FIG. 2 is an enlarged sectional view of an essential part showing a part taken out of an endoscope unit in section in the endoscope system in FIG. 1;

FIG. 3 is an outline configuration view showing an outline of an entire configuration when the endoscope system in FIG. 1 is used as a TV camera for observation of an operated portion;

FIG. 4 is an enlarged sectional view of an essential part showing apart of the camera unit in FIG. 3;

FIG. 5 is a longitudinal sectional view showing an outline configuration of a camera unit in an endoscope system of a second embodiment of the present invention;

FIG. 6 is a longitudinal sectional view showing an outline configuration of a camera unit in an endoscope system of a third embodiment of the present invention;

FIG. 7 is a longitudinal sectional view showing an outline configuration of a camera unit in an endoscope system of a fourth embodiment of the present invention when a distance to an observation portion (WD1) is long;

FIG. 8 is a longitudinal sectional view showing an outline configuration of a camera unit in the endoscope system of the fourth embodiment of the present invention when a distance to an observation portion (WD2) is shorter than the case of FIG. 7 (WD1);

FIG. 9 is an outline configuration view of an endoscope unit in the endoscope system of the fourth embodiment of the present invention;

FIG. 10 is a longitudinal sectional view showing an outline configuration of a camera unit in an endoscope system of a fifth embodiment of the present invention when a distance to an observation portion (WD1) is long;

FIG. 11 is a longitudinal sectional view showing an outline configuration of a camera unit in the endoscope system of the fifth embodiment of the present invention when a distance to an observation portion (WD2) is shorter than the case of FIG. 10 (WD1);

FIG. 12 is a longitudinal sectional view showing an outline configuration of a camera unit in an endoscope system of a sixth embodiment of the present invention;

FIG. 13 is a longitudinal sectional view showing an outline configuration of an endoscope unit in the endoscope system of the sixth embodiment of the present invention;

FIG. 14 is a longitudinal sectional view showing an outline configuration of a camera unit in an endoscope system of a seventh embodiment of the present invention;

FIG. 15 is a longitudinal sectional view showing an outline configuration of an endoscope unit in the endoscope system of the seventh embodiment of the present invention;

FIG. 16 is a longitudinal sectional view showing an outline configuration of a camera unit in an endoscope system of a eighth embodiment of the present invention;

FIG. 17 is a longitudinal sectional view showing an outline configuration of an endoscope unit in the endoscope system of the eighth embodiment of the present invention;

FIG. 18 is a longitudinal sectional view showing an outline configuration of a camera unit in an endoscope system of a ninth embodiment of the present invention;

FIG. 19 is a longitudinal sectional view showing an outline configuration of an endoscope unit in the endoscope system of the ninth embodiment of the present invention;

FIG. 20 is a longitudinal sectional view showing an outline configuration of a camera unit in an endoscope system of a tenth embodiment of the present invention;

FIG. 21 is a longitudinal sectional view showing an outline configuration of an endoscope unit in the endoscope system of the tenth embodiment of the present invention;

### DESCRIPTION OF THE PREFERRED EMBODIMENTS

The present invention will be described below referring to illustrated embodiments.

### First Embodiment

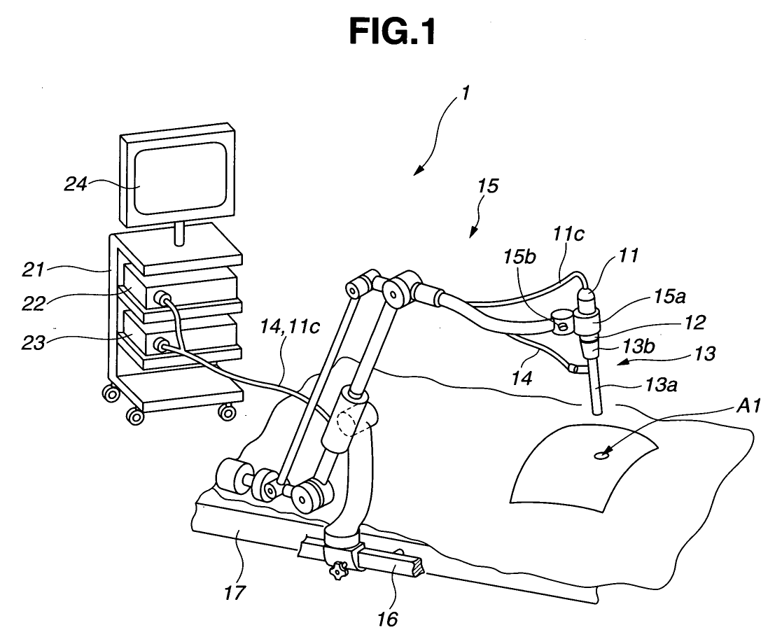

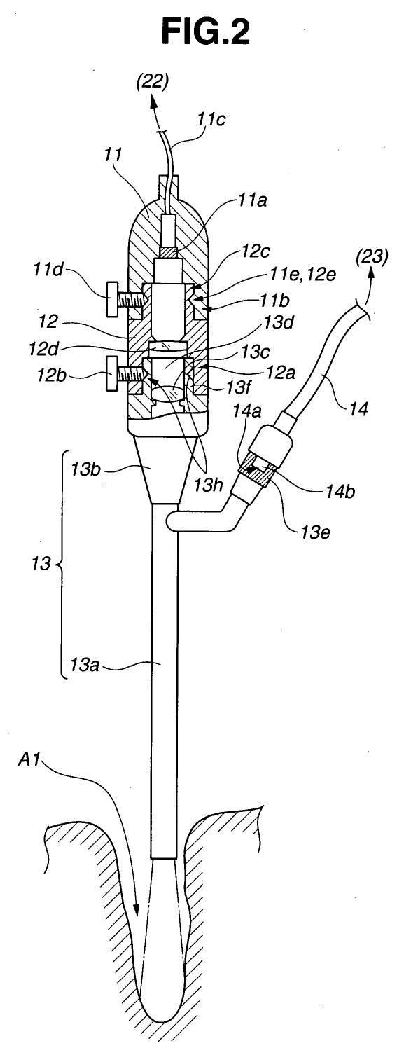

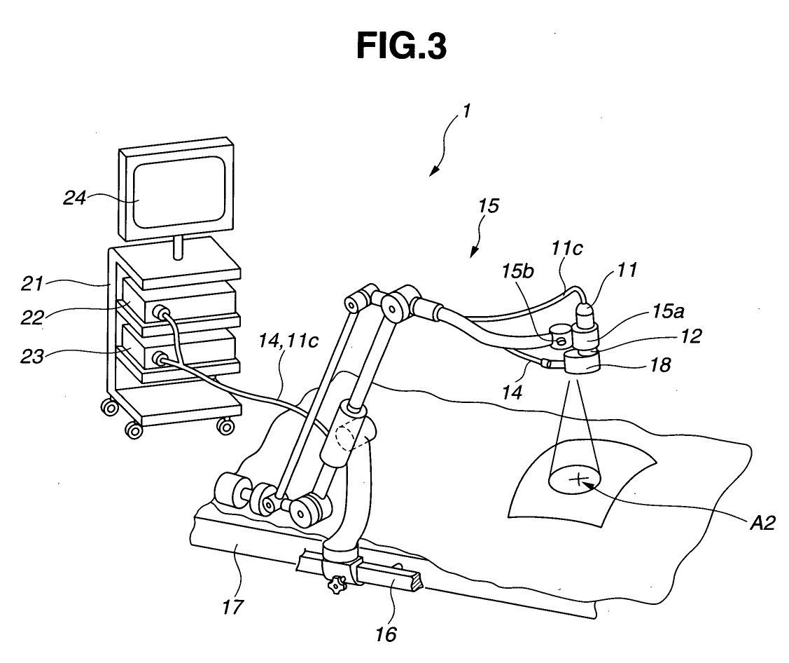

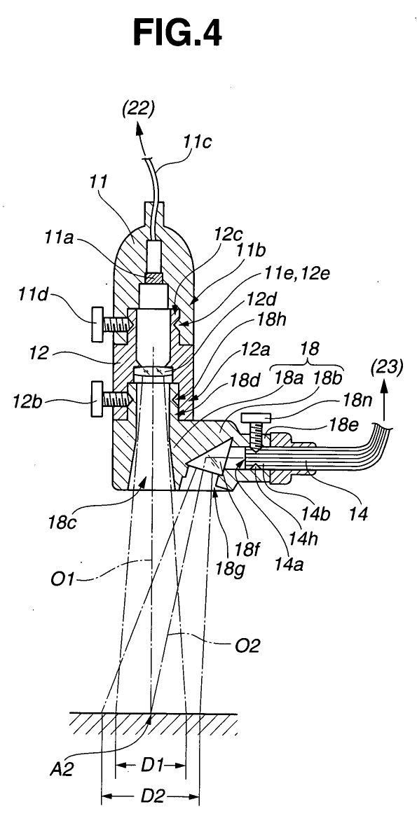

FIGS. 1 to 4 are views showing an endoscope system of a first embodiment of the present invention. Among them, FIGS. 1 and 2 are views showing a state where an endoscope system of this embodiment is used as an endoscope. FIGS. 3 and 4 are views showing a state where the endoscope of this embodiment is used as a TV camera for observing an operated portion.

More particularly, FIG. 1 is an outline configuration view showing an outline of an entire configuration when the endoscope system of this embodiment is used as an endoscope. FIG. 2 is an enlarged sectional view showing an essential part taken out of an endoscope unit in section in the endoscope system of FIG. 1. FIG. 3 is an outline configuration view showing an outline of an entire configuration when the endoscope system in FIG. 1 is used as a TV camera for observing an operated portion. FIG. 4 is an enlarged sectional view showing an essential part in section taken out of a camera unit in FIG. 3.

As shown in FIG. 1, an endoscope system I of this embodiment comprises image pickup means including a camera head 11, which is an image pickup apparatus provided with an image pickup device 11a (See FIG. 2) and the like inside and a camera adapter 12 connected to this camera head 11, an endoscope 13 connected to this camera adapter 12, a light guide 14 connected to this endoscope 13, a holder portion 15 for fixing and holding a configuration unit to which each of the camera head 11, the camera adapter 12 and the endoscope 13 is integrally connected in a predetermined space, a rail portion 16 for holding this holder portion 15 movable in a predetermined direction and fixing it at a predetermined portion, a bed 17 on which this rail portion 16 is fixed and held and a subject is loaded, a camera control unit (hereinafter referred to as CCU) 22 for controlling the camera head 11, a light source device 23, which is an illumination light source to which the other end of the light guide 14 is connected and ejects an illumination light, a monitor 24, which is a display device for displaying an observation image taken in by the endoscope 13 and converted by the camera head 11 to an electric signal, and a trolley 21 on which constituent equipment such as the CCU 22, the light source device 23, and the monitor 24 are mounted.

As shown in FIGS. 1 and 2, the endoscope 13 in the endoscope system 1 of this embodiment comprises an elongated insertion portion 13a to be inserted into a body cavity and a base end 13b connected to the base end side of this insertion portion 13a.

In the insertion portion 13a, an objective optical system, a relay optical system and the like are incorporated (any of them not shown). Also, in the base end 13b, an ocular lens 13c is incorporated.

At the base end 13b, an optical image ejection portion 13d from which an optical image formed by a luminous flux transmitted through the ocular lens 13c is ejected is opened to the rear of the ocular lens 13c.

By this, an image of the subject formed by the objective optical system at the tip end of the insertion portion 13a, that is, light from the subject guided through the insertion portion 13a is transmitted to the ocular lens 13c of the base end 13b through the relay optical system and after having transmitted through this ocular lens 13c, ejected toward the rear of the ocular lens 13c and forms an image at a predetermined position.

Also, at a predetermined portion on the outer circumferential face on the base end side of the insertion portion 13a, a light-guide connection portion (hereinafter referred to as LG connection portion) 13e, which is an illumination light incident portion for guiding the illumination light from the light guide 14, which is the illumination light ejecting means, is extended toward the outside. To this LG connection portion 13e, an illumination light ejection portion 14a formed at one end of the light guide 14 is detachably connected. Thus, in the vicinity of the illumination light ejection portion 14a of the light guide 14, a second mounting portion 14b is formed, which is a second connection portion for surely attaching the LG connection portion 13e.

That is, the second mounting portion 14b, which is the second connection portion, is provided at the light guide 14 (illumination light ejecting means) for attaching the LG connection portion 13e of the endoscope 13 to the light guide 14 (illumination light ejecting means) so that the illumination light ejected by the illumination light ejection portion 14a can enter the LG connection portion 13e (illumination light incident portion).

By fitting the second mounting portion 14b of the light guide 14 to the LG connection portion 13e, the light guide 14 can be attached to the endoscope 13 and both can be connected to each other.

When the both are connected to each other, the illumination light from the illumination light ejection portion 14a of the light guide 14 is ejected toward the LG connection portion 13e and enters the relay optical system inside the insertion portion 13a of the endoscope 13.

On the other hand, the other end of the light guide 14 is connected to the light source device 23 mounted on the trolley 21, which will be described later. By this, the illumination light ejected from the light source device 23 is supplied to the endoscope 13 through the light guide 14 and the LG connection portion 13e and further guided to the tip end of the insertion portion 13a through the relay optical system (not shown) penetrating through the insertion portion 13a of the endoscope 13 and then, ejected from the tip end face to the front so that the illumination light illuminates a desired observed portion (operated portion) Al (See FIGS. 1 and 2).

To the base end 13b of the endoscope 13, one end (tip end side) of the camera adapter 12 is detachably connected. For that purpose, a camera connection portion 13f is formed in the vicinity of the optical image ejection portion 13d at the base end 13b of the endoscope 13.

This camera connection portion 13f attaches the endoscope 13 to one end of the camera adapter 12 constituting a part of the image pickup means so that the luminous flux from the subject ejecting from the optical image ejection portion 13d through the objective optical system and the relay optical system of the endoscope 13 can be captured by the image pickup device 11a, which will be described later. That is, this camera connection portion 13f is formed capable of being fitting or optionally being detachably attached to a first mounting portion 12a (which will be described later), which is a first connection portion provided at a body portion of the camera adapter 12.

The camera adapter 12 is formed having the body portion substantially in the cylindrical shape as shown in FIG. 2. At one end of this body portion (tip end side), the first mounting portion 12a, which is the above first connection portion, is provided, while a camera mounting portion 12c is provided at the other end (base end side) of the body portion.

Inside the body portion of the camera adapter 12, an image forming lens 12d is disposed for transmitting the luminous flux from the ocular lens 13c of the base end portion 13b of the endoscope 13 and further ejecting it toward the image pickup device 11a (which will be described later) disposed at the rear so as to form an image at a predetermined position (light receiving surface of the image pickup device 11a). For that purpose, the ocular lends 13c of the endoscope 13 and the image forming lens 12d of the camera adapter 12 are disposed coaxially so that their optical axes approximately match each other. Also, the optical axes are set in the direction substantially orthogonal to the light receiving surface of the image pickup device 11a to pass through substantially the center part of the light receiving surface of the image pickup device 11a.

At the above-mentioned first mounting portion 12a, a first fixing member 12b is disposed. This first fixing member 12b is disposed so as to penetrate the first mounting portion 12a in the direction orthogonal to the axial direction of the camera adapter 12. The axial direction of the camera adapter 12 here means the direction along the optical axis of the luminous flux incident to the light receiving surface of the image pickup device 11a after transmitting through the image forming lens 12d.

The first fixing member 12b is formed by a male screw, for example, and at a predetermined portion of the first mounting portion 12a corresponding to that, a female screw penetrating the first mounting portion 12a in the direction orthogonal to its axial direction is formed. And the male screw portion of the first fixing member 12b is screwed with the female screw portion of the first mounting portion 12a.

When the first mounting portion 12a of the camera adapter 12 and the camera connection portion 13f of the base end 13b of the endoscope 13 are fitted with each other as shown in FIG. 2, an engagement peripheral groove 13h with a section in the substantially V-shaped is formed at a portion opposite to an opening of the female screw portion and on the outer circumferential face of the camera connection portion 13f.

Therefore, while the camera connection portion 13f of the endoscope 13 is fitted to the first mounting portion 12a of the camera adapter 12, if the first fixing member 12b is screwed with the female screw portion of the first mounting portion 12a and rotated in the tightening direction, the tip end of the first fixing member 12b penetrates the first mounting portion 12a and is fitted with the engagement peripheral groove 13h on the outer circumferential face of the camera connection portion 13f. By this, the endoscope 13 is brought into the state that it can not be pulled out of the camera adapter 12. Thus, the endoscope 13 is fixed to the camera adapter 12. The tip end of the first fixing member 12b has a section in the substantially V-shape conforming to the sectional shape of the engagement peripheral groove 13h fitted with it. By this, when both are fitted with each other, the camera adapter 12 and the endoscope 13 are relatively positioned.

Also, while the both are in the fixed state, if the first fixing member 12b is rotated in the loosening direction, the fitted state between the tip end of the first fixing member 12b and the engagement peripheral groove 13h of the camera connection portion 13f is disengaged. Therefore, if the endoscope 13 is pulled out of the camera adapter 12 in this state, the connected state between the camera adapter 12 and the endoscope 13 can be released. By this, the endoscope 13 can be removed from the camera adapter 12.

To the other end (base end side) of the body portion of the camera adapter 12, the camera head 11 is optically and detachably connected. For that purpose, a connection portion 11b is formed at one end (tip end side) of the camera head 11. And the camera mounting portion 12c of the body portion of the camera adapter 12 is fitted with this connection portion 11b.

In this case, on the inner circumferential face of the connection portion 11b, an engagement projecting portion 11e with a section in the substantially V-shape and projecting inward is formed. In correspondence with this, an engagement groove portion 12e with a section in the substantially V-shape is formed on the outer circumferential face of the camera mounting portion 12c.

When the connection portion 11b is to be fitted with the camera mounting portion 12c, the camera mounting portion 12c is fitted to the connection portion 11b, and the camera mounting portion 12c is pushed in. Then, the tip end edge portion of the camera mounting portion 12c is brought into contact with the engagement projecting portion 11e. By further adding a force amount in the direction to push the camera adapter 12 to the camera head I1, the connection portion 11b is slightly deflected outward and the opening side of the connection portion 11b is pushed wide. By this, the camera mounting portion 12c can be pushed in. And when the engagement projecting portion 11e is engaged with the engagement groove portion 12e, the deflection of the connection portion 11b is cancelled. At this position, the camera head 11 and the camera adapter 12 are relatively positioned. At the same time, the fitted state of the both is held with a predetermined force amount.

Also, a second fixing member 11d is disposed at the connection portion 11b of the camera head 11. This second fixing member 11d is configured substantially similarly to the above-mentioned first fixing member 12b and is formed by a male screw, for example. In correspondence with that, a female screw penetrating the connection portion 11b in the direction orthogonal to the axial direction is provided at a predetermined portion of the connection portion 11b. The male screw portion of the second fixing member 11d is screwed with the female screw portion of the connection portion 11b.

Therefore, while the camera mounting portion 12c of the camera adapter 12 is fitted to the connection portion 11b of the camera head 11, if the second fixing member 11d is screwed with he female screw portion of the connection portion 11b and rotated in the tightening direction, the tip end of the second fixing member 11d is brought into contact with the outer circumferential face of the camera mounting portion 12c through the connection portion 11b. By this, the camera adapter 12 can be fixed to the camera head 11. Also, when both are in the fixed state, by rotating the second fixing member 11d in the loosening direction so as to release the contact state between the tip end of the second fixing member 11d and the outer circumferential face of the camera mounting portion 12c, connection of the camera adapter 12 with the camera head 11 can be released, by which the camera adapter 12 can be removed from the camera head 11.

The camera head 11 is connected to the CCU 22 mounted on the trolley 21 through a cable 11c. To this CCU 22, the monitor 24 mounted on the trolley 21 is connected.

By this, an observation image taken in by the endoscope 13 is inputted to the camera head 11 through the camera adapter 12. The inputted observation image is given photoelectric conversion processing and converted by the camera head 11 to an electric signal in a predetermined form. The electric signal generated by the camera head 11 is transmitted to the CCU 22 through the cable 11c. Upon receiving it, the CCU 22 generates a video signal for display and transmits it to the monitor 24. Upon receiving it, the monitor 24 displays the observation image based on the video signal.

A unit (hereinafter referred to as an endoscope unit) in which the camera head 11, the camera adapter 12, the endoscope 13 and the light guide 14 are integrally connected is held by a holding portion 15a disposed at the tip end of the holder portion 15.

The holder portion 15 comprises a plurality of support arm members and a link mechanism made of articulate members or the like formed rotatably in the vertical direction or the horizontal direction by connecting these support members.

At the holding portion 1Sa of the holder portion 15, an arm lock/unlock switch 15b is disposed in the vicinity of its base end. This arm lock/unlock switch 15b switches the state of the support arm between a locked state and an unlocked state.

An operator brings the support arm state into the unlocked state and then, the operator can dispose the holding portion 15a of the holder portion 15 and a unit held by that (endoscope unit or a camera unit, which will be described later) arbitrarily at a desired position in the space. And in order to hold the position, by locking the state of the support arm by the arm lock/unlock switch 15b, the holding portion 15a of the holder portion 15 and the unit held by that can be fixed and held at the desired spatial position.

Since this holder portion 15 is not directly related to the present invention, detailed description of its configuration will be omitted.

In the endoscope system 1 of this embodiment configured as above, the endoscope 13 is made detachable with respect to the camera adapter 12 as mentioned above.

That is, in the endoscope system 1 of this embodiment, by releasing the connection between the camera adapter 12 and the endoscope 13 and by releasing the connection between the endoscope 13 and the light guide 14, the endoscope 13 can be fully removed from the camera adapter 12.

In this state, in this endoscope system 1, the image pickup means configured by the camera head 11 and the camera adapter 12 can be used as a television camera (TV camera) for observing an operated portion.

In this case, it is very convenient if the illumination light of the light guide 14 can irradiate the desired observation portion, that is, the image pickup region by the image pickup means.

Then, the endoscope system I of this embodiment, the light guide 14 is fixed and held and an illumination adapter 18 is provided, which is an adapter provided with a prism 18f (which will be described later. See FIG. 4), which is an illumination optical system for guiding the illumination light ejected form this light guide 14 to an image pickup region D1 (See FIG. 4) by the image pickup means and an illumination optical path changing optical system. And this illumination adapter 18 is configured detachable with respect to the image pickup means so that the illumination adapter 18 can be attached instead of the above endoscope 13.

This illumination adapter 18 is attached to the camera adapter 12 instead of the endoscope 13 and configured so that it can be attached to the camera adapter 12 by the same attachment/detachment means as that of the endoscope 13 at the same portion as that of the endoscope 13.

Also, to this illumination adapter 18, the light guide 14 is connected and fixed at a predetermined portion. In this case, the connection between the illumination adapter 18 and the light guide 14 is carried out by attachment/detachment means configured in the same way as the attachment/detachment means between the endoscope 13 and the camera adapter 12 or the attachment/detachment means between the illumination adapter 18 and the camera adapter 12 (the detail will be described later).

The unit configured by attaching the illumination adapter 18 to which the light guide 14 is connected to the image pickup means made of the camera head 11 and the camera adapter 12 (hereinafter referred to as a camera unit) can be used as a TV camera for observing an operated portion.

The configuration of camera unit in this case, that is, the unit comprised by the camera head 11, the camera adapter 12, the illumination adapter 18 and the light guide 14 will be described below using FIGS. 3 and 4.

This illumination adapter 18 is formed by the body portion 18a in the substantially cylindrical shape having a through hole 18c inside as shown in FIG. 4 and a projecting portion 18b projected outward from this body portion 18a.

In the vicinity of one of openings of the through hole 18c drilled in the body portion 18a of the illumination adapter 18, a first adapter connection portion 18d, which is a first adapter connection portion for connecting the illumination adapter 18 and the camera adapter 12 to each other is formed.

This first adapter connection portion 18d is formed substantially in the same shape as that of the above-mentioned camera connection portion 13f of the endoscope 13 so that it can be fitted with the first mounting portion 12a (first connecting portion) of the camera adapter 12. Also, on the outer circumferential face of the first adapter connection portion 18d, an engagement peripheral groove 18h in the same shape as that of the above-mentioned engagement peripheral groove 13h of the camera connection portion 13f of the endoscope 13 is formed.

Therefore, mounting of the illumination adapter 18 and the camera adapter 12 can be carried out at the same time using attachment/detachment means for attaching/detaching the endoscope 13 with respect to the camera adapter 12, that is, the above-mentioned first fixing member 12b.

That is, in order to fix the first mounting portion 12a and the first adapter connection portion 18d, first, the first adapter connection portion 18d is fitted with the first mounting portion 12a. In this state, the first fixing member 12b is screwed with the female screw portion of the first mounting portion 12a and rotated in the tightening direction. Then, the tip end of the first fixing member 12b penetrates the first mounting portion 12a and is fitted with the engagement peripheral groove 18h of the first adapter connection 18d. By this, the illumination adapter 18 can not be pulled out of the camera adapter 12 any more. Thus, the illumination adapter 18 is fixed to the camera adapter 12. The tip end shape of the first mounting portion 12a is formed with a section substantially in the V-shape conforming to the sectional shape of the engagement peripheral groove 13h to be fitted with this. By this, when the both are brought into the fitted state, the camera adapter 12 and the endoscope 13 can be relatively positioned.

Also, when the both are in the fixed state, if the first fixing member 12b is rotated in the loosening direction, the fitted state between the tip end of the first fixing member 12b and the engagement peripheral grove 18h of the first adapter connection portion 18d is disengaged. Therefore, in this state, when the illumination adapter 18 is pulled out of the camera adapter 12, the connected state between the camera adapter 12 and the illumination adapter 18 can be disconnected. By this, the illumination adapter 18 can be removed from the camera adapter 12.

On the other hand, while the first adapter connection portion 18d of the illumination adapter 18 is attached to the first mounting portion 12a of the camera adapter 12, the through hole 18c is arranged in the direction along an optical axis O1 of the image forming lens 12d of the camera adapter 12 as shown in FIG. 4. By this, the through hole 18c forms an optical path through which the luminous flux incident from an observation portion by the image pickup means (camera head 11 and the camera adapter 12) or an observation portion (operated portion) A2 shown in FIG. 4, for example, is passed and made to enter the image forming lens 12d of the camera adapter 12 without obstructing the luminous flux.

On the other hand, at the tip end side of the projecting portion 18b of the illumination adapter 18, a second adapter connection portion 18e, which is a second adapter connection portion for connecting the illumination adapter 18 and the light guide 14 to each other, is provided.

This second adapter connection portion 18e is formed having the same attachment/detachment means as the attachment/detachment means between the camera adapter 12 and the endoscope 13 or the illumination adapter 18 so that the second mounting portion 14b of the light guide 14 (second connection portion) can be fitted and fixed.

That is, a fixing screw 18n is disposed at the second adapter connection portion 18e of the illumination adapter 18. This fixing screw 18n is disposed so as to penetrate the second adapter connection portion 18e in the direction orthogonal to the axial direction of the second adapter connection portion 18e of the illumination adapter 18. The axial direction of the second adapter connection portion 18e of the illumination adapter 18 is a direction along the optical axis from the illumination light ejection portion 14a to the prism 18f in an optical axis O2 ejected from the light guide 14 when the light guide 14 is attached to the second adapter connection portion 18e (See FIG. 4).

The fixing screw 18n is formed by a male screw, for example, and in correspondence with that, at a predetermined portion of the second adapter connection portion 18e, a female screw penetrating the second adapter connection portion 18e in the direction orthogonal to the axial direction is formed. And the male screw portion of the fixed screw 18n is screwed with the female screw portion of the second adapter connection portion 18e.

Also, when the second adapter connection portion 18e of the illumination adapter 18 is fitted with the second mounting portion 14b of the light guide 14 as shown in FIG. 4, at a portion opposite to the opening portion of the female screw portion and on the outer circumferential face of the second mounting portion 14b, an fengagement peripheral groove 14h with a section substantially in the V-shape is formed.

Therefore, while the second mounting portion 14b of the light guide 14 is fitted with the second adapter connection portion 18e of the illumination adapter 18, if the fixing screw 18n is screwed with the female screw portion of the second adapter connection portion 18e and rotated in the tightening direction, the tip end of the fixing screw 18n penetrates the second adapter connection portion 18e and is fitted with the engagement peripheral groove 14h on the outer circumferential face of the second mounting portion 14b. By this, the light guide 14 can not be pulled out of the illumination adapter 18 any more. Thus, the light guide 14 is fixed to the illumination adapter 18. The tip end shape of the fixed screw 18n has a section in the substantially V-shape conforming to the sectional shape of the engagement peripheral groove 14h fitted with this. By this, when the both are in the fitted state, the illumination adapter 18 and the light guide 14 are relatively positioned.

Also, when the both are in the fixed state, if the fixing screw 18n is rotated in the loosening direction, the fitted state between the tip end of the fixing screw 18n and the engagement peripheral groove 14h of the second mounting portion 14b is disengaged. Therefore, if the light guide 14 is pulled out of the illumination adapter 18 in this state, the connected state between the illumination adapter 18 and the light guide 14 can be disconnected. By this, the light guide 14 can be removed from the illumination adapter 18.

Inside the projecting portion 18b of the illumination adapter 18, the prism 18f, which is an illumination optical path changing optical system for guiding the illumination light ejected from the light guide 14 connected to the illumination adapter 18 to a desired direction is incorporated.

This prism 18f is formed by an optical prism or the like provided with an incident surface for having the illumination light from the light guide 14 enter, a reflective surface for deflecting the optical axis O2 (See FIG. 4) of the illumination light by receiving and reflecting the illumination light incident from this incident surface in a predetermined direction, and an ejecting surface for ejecting the illumination light reflected by this reflective surface in a predetermined direction.

Also, inside the projecting portion 18b of the illumination adapter 18, a predetermined space is provided at a portion along the optical axis O2 of the illumination light ejected from the illumination light ejection portion 14a between the incident surface of the prism 18f and the illumination light ejection portion 14a of the light guide 14. This space is provided so that the illumination light can enter the incident surface and forms an optical path of the illumination light.

Also, at a portion on the side opposite to the ejecting surface of the prism 18f and at a predetermined portion on the bottom surface of the illumination adapter 18, an ejecting opening 18g is provided. And inside the projecting portion 18b of the illumination adapter 18, a predetermined space is provided at a portion along the optical axis O2 of the illumination light ejected from the ejecting surface between the ejecting opening 18g and the ejecting surface. This space is provided so that the illumination light can be ejected outward from the ejecting surface and forms an optical path of the illumination light.

In this way, in the state where the second mounting portion 14b of the light guide 14 (second connection portion) is connected to the second adapter connection portion 18e of the illumination adapter 18, the light guide 14 is surely fixed and supported by the illumination adapter 18 as shown in FIG. 4. At the same time, when the both (illumination adapter 18 and the light guide 14) are connected to each other, the illumination light ejected from the light guide 14 is ejected from the ejecting opening 18g through the optical path and the prism 18f in the illumination adapter 18.

In this case, the illumination light incident to the illumination adapter 18 has the optical axis O2 deflected by the prism 18f and ejected from the ejecting opening 18g and then, the illumination light is ejected toward the observation portion, which is an image pickup region D1 by the camera head 11 and the camera adapter 12. By this, the observation portion, that is, the predetermined portion (surface of the operated portion) A2 on the subject whose image is to be captured can be surely illuminated.

The reflective surface of the prism 18f is set so that the illumination light incident from the incident surface can be ejected toward the image pickup region D1 by the image pickup means through the ejecting surface.

That is, it is set so that the optical axis O1 and the optical axis O2 cross each other at a predetermined portion, in the vicinity of the substantially center part of the image pickup region D1, for example, at the observation portion (surface of the operated portion) shown by reference character A2 in FIG. 4, for example. By this, since an illuminating region D2 by the illumination light can be overlapped with the image pickup region D1, the desired observation portion can be surely illuminated.

In other words, the illumination adapter 18 is a constituent member for guiding the illumination light from the light guide 14 so that the center of the illumination light axis of the light guide 14 connected to the illumination adapter 18 substantially matches the center of the image pickup optical axis of the camera head 11 connected through the camera adapter 12 and for fixing and holding the light guide 14 so as to maintain that state.

An outline of action when the endoscope system I of this embodiment configured in this way is as follows.

First, the camera unit in the state as shown in FIGS. 3 and 4, that is, comprised by attaching the illumination adapter 18 to which the light guide 14 is connected to the image pickup means (camera head 11 and the camera adapter 12) is used as a TV camera for observation of an operated portion for performing an incision treatment or the like before an operation.

In this case, an operator arranges the camera unit shown in FIG. 4 at a spatial position where a desired portion on a subject (vicinity of the surface of the operated portion) can be illuminated and observed. For that purpose, first, the operator unlocks the holder portion 15 by operating the arm lock/unlock switch 15b so as to make it movable. And the camera unit held by the holding portion 15a of the holder portion 15 is arbitrarily moved and arranged at the desired spatial position. By operating the arm lock/unlock switch 15b again, the holder portion 15 is brought into the locked state. By this, the camera unit is fixed and held at the desired spatial position.

If the CCU 22 and the light source device 23 are started in this state, the image pickup operation by the camera head 11 in this camera unit is started, and the illumination light by the light source device 23 illuminates the desired portion from the light guide 14 through the illumination adapter 18, that is, the observation portion (surface of the operated portion) A2 in FIGS. 3 and 4. By this, an image of a predetermined image pickup range around the portion A2 is displayed on the monitor 24. The operator performs pre-operation procedure or the like while looking at the image displayed on the monitor 24.

When the pre-operation procedure is finished, then, the operator performs an operation using the endoscope 13.

For that purpose, first, the operator removes the illumination adapter 18 from the camera unit in the state shown in FIGS. 3 and 4 and attaches the endoscope 13 to the camera adapter 12 so as to switch to the endoscope unit form shown in FIGS. 1 and 2.

In order to remove the illumination adapter 18 from the camera unit, after loosing the first fixing member 12b, the illumination adapter 18 is pulled out, for example, of the camera adapter 12.

Also, the light guide 14 is removed from the illumination adapter 18 and the light guide 14 is connected to the endoscope 13. In order to remove the light guide 14 from the illumination adapter 18, after loosing the fixing screw 18n, the light guide 14 is pulled out, for example, from the illumination adapter 18.

And the endoscope 13 is attached to the camera adapter 12 in place of the illumination adapter 18, and the first fixing member 12b is operated in the tightening direction. Then, the light guide 14 is fitted with the LG connection portion 13e of the endoscope 13 and attached.

In this way, the endoscope unit in the state shown in FIGS. 1 and 2 is formed.

Next, the operator operates the arm lock/unlock switch 15b so as to unlock the holder portion 15. And the endoscope 13 of the endoscope unit is moved toward the operated portion Al and the insertion portion 13a is inserted into a body cavity of the subject. At this time, an endoscopic image by the endoscope 13 is displayed on the monitor 24. The operator performs a predetermined operational procedure while looking at the endoscopic image displayed on the monitor 24.

When the predetermined operational procedure is finished, the operator performs a suture procedure of the incision portion or the like. For that purpose, the endoscope unit is switched to the camera unit form again. In order to remove the endoscope 13 from the endoscope unit, predetermined operations such as loosening the first fixing member 12b are performed. They are substantially the same procedures to the above-mentioned one to remove the illumination adapter 18 from the camera unit.

Also, the light guide 14 is removed from the endoscope 13 and the light guide 14 is connected to the illumination adapter 18. In order to remove the light guide 14 from the endoscope 13, an operation to release the fitted state between the LG connection portion 13e of the endoscope 13 and the second mounting portion 14b (second connection portion) of the light guide 14, that is, a pulling-out operation, for example, is performed.

Then, the illumination adapter 18 is attached to the camera adapter 12, and the illumination adapter 18 is brought into the fixed state by operation such as tightening the first fixing member 12b or the like. Also, the second mounting portion 14b (second connection portion) of the light guide 14 is connected to the second adapter connection portion 18e of the illumination adapter 18, and the fixing screw 18n is tightened.

In this way, the camera unit in the state shown in FIGS. 3 and 4 is formed.

In this state, an image pickup operation is started using the camera unit. At this time, the illumination light from the light guide 14 illuminates the predetermined observation portion A2 through the illumination adapter 18 (FIGS. 3 and 4). On the monitor 24, an image in the predetermined image pickup range around the portion A2 is displayed. The operator performs the suture procedure or the like of the incised portion while looking at the image displayed on the monitor 24.

As mentioned above, according to the above first embodiment, since the first fixing member 12b is interposed between the first mounting portion 12a of the camera adapter 12 and the camera connection portion 13f of the endoscope 13 (See FIGS. 1 and 2) or between the first mounting portion 12a of the same and the first adapter connection portion 18d of the illumination adapter 18 (See FIGS. 3 and 4), it is so configured that either of the endoscope 13 or the illumination adapter 18 can be detachably disposed at the camera adapter 12.

And when the camera adapter 12 is connected to the endoscope 13 (state shown in FIGS. 1 and 2), the light guide 14 is connected to the LG connection portion 13e of the endoscope 13, while when the camera adapter 12 is connected to the illumination adapter 18 (state shown in FIGS. 3 and 4), the light guide 14 is connected to the second adapter connection portion 18e of the illumination adapter 18.

By this, in the state shown in FIGS. 1 and 2, after the endoscope 13 is removed from the camera adapter 12 and the light guide 14 is removed from the endoscope 13, the first mounting portion 12a (first connection portion) of the camera adapter 12 can be connected to the first adapter connection portion 18d of the illumination adapter 18 using the same attachment/detachment means (first fixing member 12b).

Moreover, by connecting the second mounting portion 14b of the light guide 14 to the second adapter connection portion 18e of the illumination adapter 18 using the same attachment/detachment means as that between the illumination adapter 18 and the camera adapter 12, the state shown in FIGS. 3 and 4 can be obtained.

Therefore, when the endoscope 13 is connected to the image pickup means (camera head 11 and the camera adapter 12), it can be used as the endoscope device, while when the endoscope 13 is removed from the image pickup means (11, 12) and the illumination adapter 18 is connected to the same image pickup means (11, 12), it can be used as the TV camera for observation of the operated portion.

In this case, since the optical axis O2 of the illumination light ejected from the light guide 14 connected to the illumination adapter 18 is set so as to cross the optical axis O1 of the image forming lens 12d of the camera adapter 12 at a predetermined portion, the range in the image pickup region by the image pickup means can be illuminated bright all the time and a favorable visual field can be ensured easily.

### Second Embodiment

A three-dimensional video observing apparatus comprising right and left pair of image pickup optical systems has been put into practice so as to make three dimensional (3D) observation of an operated portion in a three-dimensional video.

An endoscope system of a second embodiment of the present invention, which will be described below, is an example of configuration that the 3D observation can be made when it is used as a TV camera for observation of the operated portion.

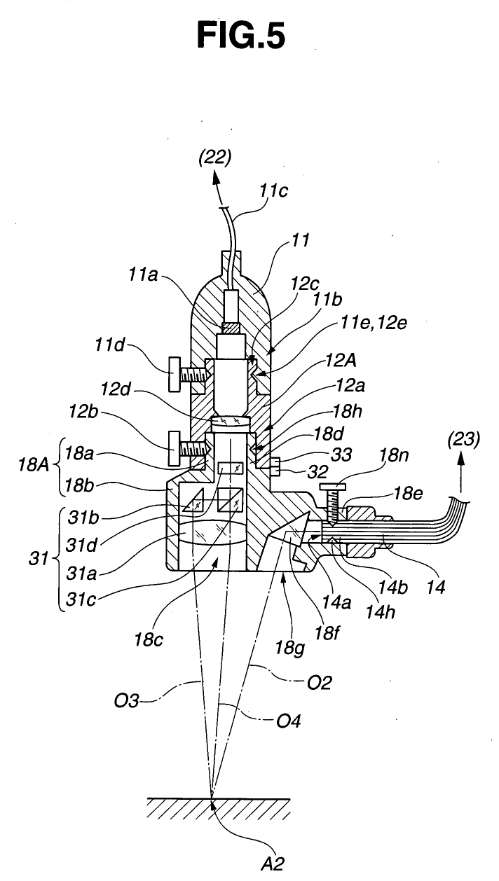

FIG. 5 is a longitudinal sectional view showing an outline configuration of a camera unit in the endoscope system of the second embodiment of the present invention.

The configuration of this embodiment is substantially the same as that of the endoscope system of the above-mentioned first embodiment basically only with a difference in the configuration of the illumination adapter constituting the camera unit and its periphery. That is, as shown in FIG. 5, they are different in the point that a 3D observation optical system 31 (31a, 31b, 31c, 31d) inside an illumination adapter 18A is provided.

Therefore, the same configuration as that of the above-mentioned first embodiment is given the same reference numeral and the detailed description will be omitted, while only the difference will be described.

Among members constituting the camera unit used in the endoscope system of this embodiment, an illumination adapter 18A incorporates, as shown in FIG. 5, the 3D observation optical system 31.

The 3D observation optical system 31 comprises an objective lens 31a, which is an objective optical system, a polarization triangular prism 31b, a polarization half prism 31c, a polarization shutter 31d, and the like.

The polarization triangular prism 31b and the polarization half prism 31c are polarizing means for polarizing at least two different luminous fluxes from the objective lens 31a into different directions.

Also, the polarization half prism 31 c is an optical system for guiding at least two different luminous fluxes to the same optical path. For example, the polarization half prism 31 c guides the two different luminous fluxes of the luminous flux from the objective lens 31a and the luminous flux from the polarization triangular prism 31b to the same optical path toward the image pickup device 1 la as shown in FIG. 5.

The polarization shutter 31d is polarization luminous flux transmitting means for alternately transmitting at least two luminous fluxes with different polarized state, that is, each of the two luminous fluxes guided to the same optical path by the polarization half prism 31 c.

By this configuration, an observation image for the right eye enters the polarization shutter 31d through the objective lens 31a, the polarization triangular prism 31b, and the polarization half prism 31 c and then, transmits through the image forming lens 12d of the camera adapter 12 and forms an image on the light receiving surface of the image pickup device 11a of the camera head 11.

Also, the observation image for the left eye enters the polarization shutter 31d through the objective lens 31a and the polarization half prism 31c. After that, as with the observation image for the right eye, an image is formed on the light receiving surface of the image pickup device 11a through the image forming lens 12d.

The polarization shutter 31d is configured to switch between a state for transmitting only a light beam O3 forming the observation image for the right eye (See FIG. 5) and a state for transmitting only a light beam O4 forming the observation image for the left eye.

Also, in this embodiment, connection terminals 32, 33 are disposed at each of the illumination adapter 18A and a camera adapter 12A. These connection terminals 32, 33 electrically connect the both (12A, 18A) when the illumination adapter 18A is attached to the camera adapter 12A, that is, a form of the camera unit is configured. And the terminal connector 33 on the camera adapter 12 side is electrically connected to the CCU 22 (See FIG. 1) through the cable 11c.

Therefore, by attaching the illumination adapter 18A to the camera adapter 12A at a normal position and electrically connecting the connection terminals 32, 33, the camera unit and the CCU 22 are electrically connected.

Also, though not shown particularly, a driving circuit for driving the polarization shutter 31d is provided at the CCU 22. By operating the polarization shutter 31 d by this driving circuit, an image for the right eye and an image for the left eye can be formed alternately on the light receiving surface of the image pickup device 11a.

The CCU 22 alternately captures an image for the right eye and an image for the left eye through driving control of the image pickup device 11a by synchronizing it to switching driving of the polarization shutter 31d by this driving circuit.

In correspondence with that, in this embodiment, a 3D display device is applied to the monitor 24 (See FIG. 1) corresponding to 3D observation.

Therefore, the CCU 22 generates an image signal for display by executing various signal processing for the image signal for the right eye and the image signal for the left eye captured and obtained as above and outputs it to the monitor 24.

Upon receiving it, the monitor 24 as the 3D display device displays the image for the right eye and the image for the left eye. By this, the 3D observation of an operated portion can be made.

The other configurations are the same as those of the above-mentioned first embodiment. The attachment/detachment means between the camera adapter 12A and the illumination adapter 18A is configured totally the same as the above-mentioned first embodiment by the first fixing member 12b and the engagement peripheral groove 18h.

Therefore, action at change from the state of the camera unit form to the endoscope unit form, that is, the procedure to remove the illumination adapter 18A from the camera adapter 12A and mounting the endoscope 13 to the camera adapter 12A or the procedure to remove the light guide 14 from the illumination adapter 18A and mounting it to the endoscope 13 are totally the same as those of the above-mentioned first embodiment.

As mentioned above, the second embodiment is the same as the above-mentioned first embodiment. In addition, in this embodiment, the objective lens 31a, the polarization triangular prism 31b, the polarization half prism 31c, the polarization shutter 31d and the like are provided inside the illumination adapter 18A so that a three-dimensional observation can be easily made for an observation image of an operated portion when used in the camera unit form.

### Third Embodiment

Next, an endoscope system of a third embodiment of the present invention will be described below.

The endoscope system of this embodiment is an example of configuration that an image pickup region when used as a TV camera for observation of an operated portion and an illumination region of the illumination light by the light guide (14) are substantially matched.

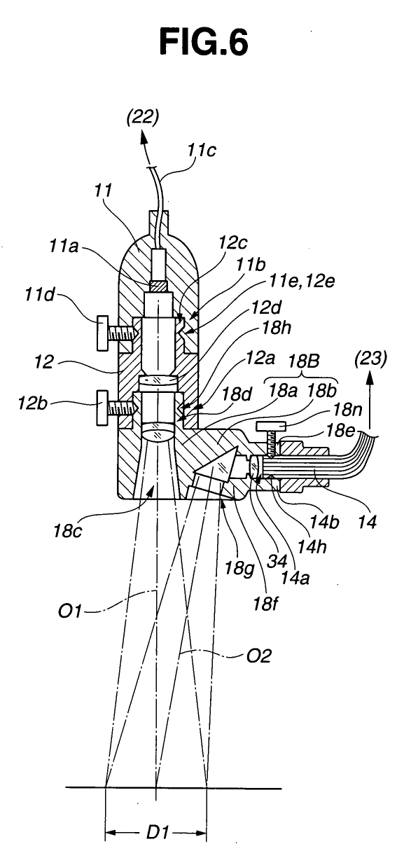

FIG. 6 is a longitudinal sectional view showing an outline configuration of a camera unit in the endoscope system of the third embodiment of the present invention.

The configuration of this embodiment is substantially the same as that of the endoscope system of the first embodiment basically and only the configuration of the illumination adapter constituting the camera unit is different. That is, as shown in FIG. 6, an illumination adapter 18B in this embodiment is different from the illumination adapter 18 in the above first embodiment in the point that it comprises a condenser lens 34, which is light condensing means for narrowing an illumination range to an illumination optical system provided inside. Therefore, the same reference numerals are given to the same configuration as that of the above first embodiment and the detailed description will be omitted but only the difference will be described.

As mentioned above, the illumination adapter 18B in members constituting the camera unit used in the endoscope system of this embodiment comprises the condenser lens 34, which is the light condensing means for narrowing the illumination range inside.

This condenser lens 34 is arranged on an optical path formed between the illumination light ejection portion 14a of the light guide 14 connected to the second adapter connection portion 18e of the illumination adapter 18B and the incident surface of the prism 18f, and its lens surface is disposed opposite to the incident surface of the above prism 18f and also to the illumination light ejection portion 14a.

By this, the illumination light ejected from the illumination light ejection portion 14a of the light guide 14 is condensed by the condenser lens 34 and then, ejected from the ejecting opening 18g through the prism 18f and illuminates a predetermined illumination region D1 (See FIG. 6).

In this case, the illumination region D1 is set so as to substantially match the image pickup region D1 by the condenser lens 34. The other configuration and action are the same as those of the above first embodiment.

As mentioned above, according to the above third embodiment, the same effect as that of the above first embodiment can be obtained. In addition, since the illumination adapter 18B further comprises the condenser lens 34 for condensing the illumination light ejected from the light guide 14 in a predetermined range so that a desired illumination region can be illuminated, when this endoscope system is used in the camera unit form, the illumination region can be set so as to substantially match the image pickup region. By this, a desired portion can be illuminated more efficiently.

### Fourth Embodiment

Next, an endoscope system of a fourth embodiment will be described below.

The endoscope system of this embodiment is an example of a case where the image pickup means comprised by a camera head and a camera adapter is provided with a focusing function and a variable power function.

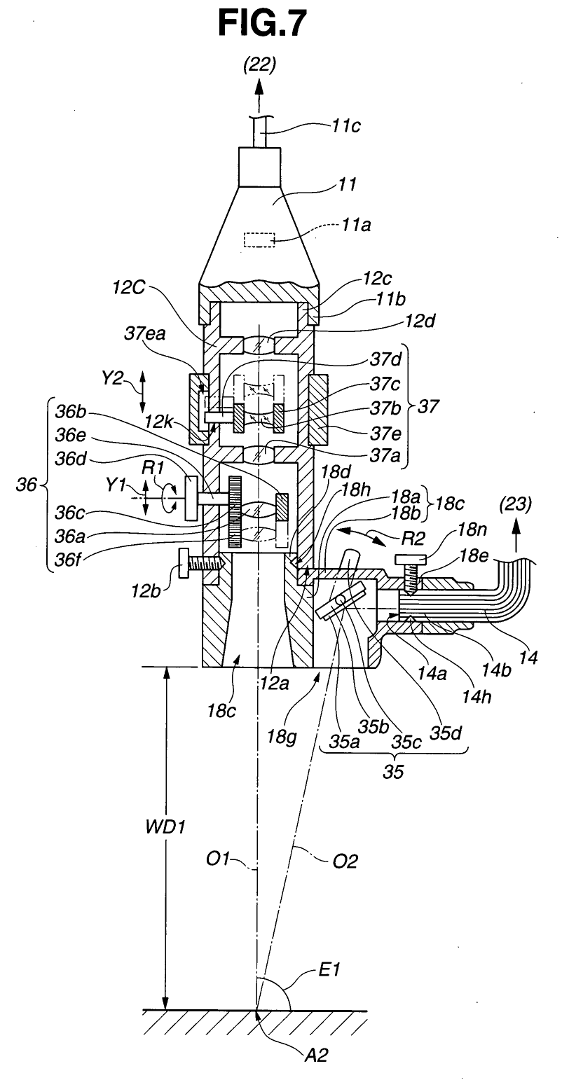

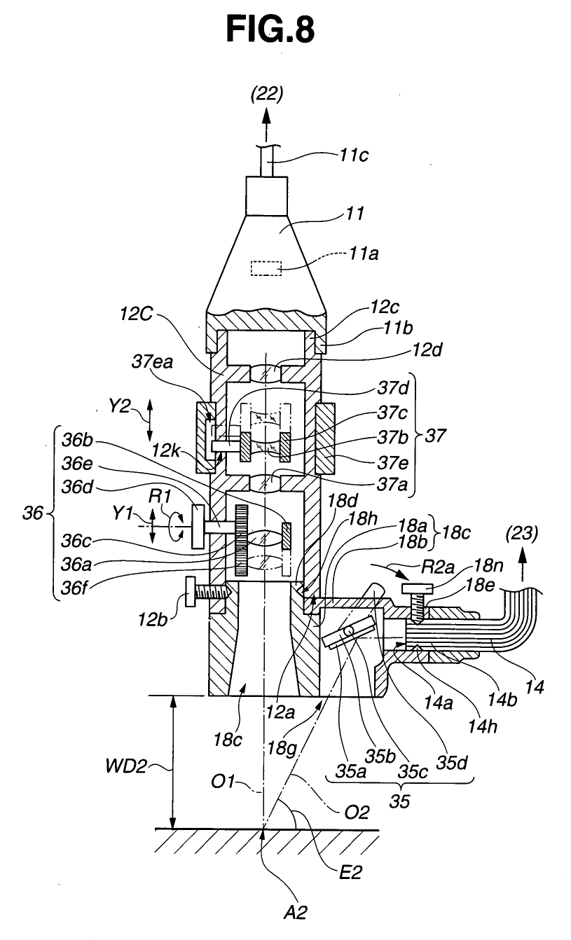

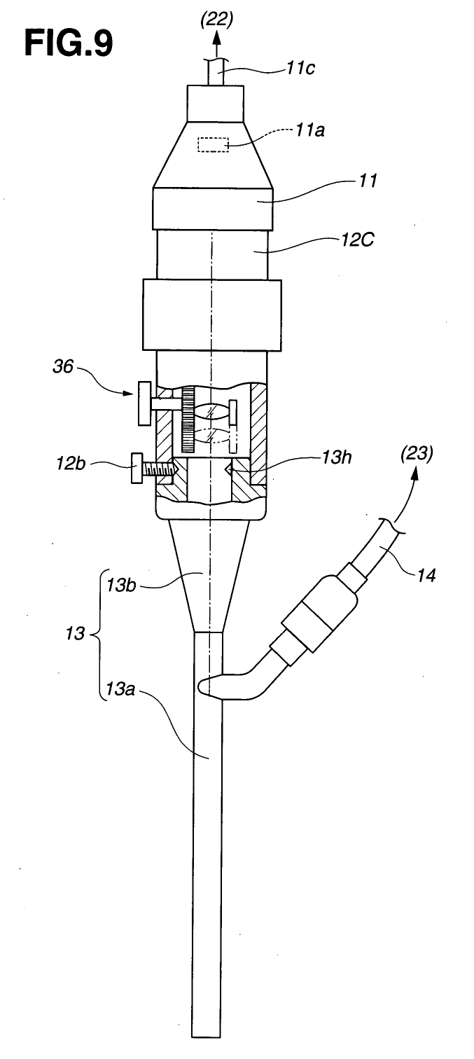

FIGS. 7, 8 and 9 show the fourth embodiment of the present invention, in which FIGS. 7 and 8 are longitudinal sectional views showing an outline configuration of a camera unit in the endoscope system of this embodiment. FIG. 7 shows an example when a distance to the observation portion (WD1) is long, while FIG. 8 shows an example when a distance to the observation portion (WD2) is shorter than the FIG. 7 case (WD1). FIG. 9 is a view showing an outline configuration of an endoscope unit in the endoscope system of this embodiment. In FIG. 9, a part of the view is shown in a section.

The configuration of this embodiment is substantially the same as that of the endoscope system of the above first embodiment basically, but it is slightly different in the configuration provided with the focusing function and the variable power function of the image pickup means, that is, the internal configuration of a camera adapter 12C and the internal configuration of an illumination adapter 18C. Therefore, the same configuration as that of the above first embodiment is given the same reference numeral and the detailed description will be omitted but only the difference will be described.

As mentioned above, the camera adapter 12C in the members constituting the image pickup means used in the endoscope system of this embodiment is provided with a focusing mechanism 36 for realizing a focusing function and a zoom mechanism 37 for realizing a variable power function inside.

The camera adapter 12C is formed having a substantially cylindrical body portion as shown in FIG. 7 similarly to that of the first embodiment. At one end (tip end side) of this body portion, the first mounting portion 12a, which is the first connection portion, is provided, while at the other end (base end side) of the body portion, the camera mounting portion 12c is provided.

Inside the body portion of the camera adapter 12C, the focusing mechanism 36, the zoom mechanism 37 for realizing the variable power function, the image forming lens 12d and the like are disposed.

The focusing mechanism 36 mainly comprises a focus lens 36a disposed movably in the direction along the optical axis O1 for focusing on the optical axis O1, a focus-lens moving frame 36b provided with a rack 36f constituting a part of a focus-lens moving mechanism for moving the focus lens 36a in the direction along the optical axis O1 and fixing/holding the focus lens 36a, a focus gear 36c meshed with the rack 36f of the focus-lens moving frame 36b and constituting a part of the focus-lens moving mechanism, a focus dial rotating shaft 36e pivotally supporting the focus gear 36c capable of rotation, and a focus dial 36d provided on the same axis of the focus dial rotating shaft 36e at the other end side and disposed outside the body portion of the camera adapter 12C.

By this configuration, when the focus dial 36d is rotated in an arrow RI direction in FIG. 7, that is, around the focus dial rotating shaft 36e, the focus gear 36c is rotated in the same direction with that. Then, the rotating force of the focus gear 36c is transmitted to the rack 36f meshed with the focus gear 36c. By this, the rack 36f is moved on the focus-lens moving frame 36b in the direction along an arrow Y1 shown in FIG. 7, that is, the direction along the optical axis O1. With this movement, the focus lens 36a fixed and held by the focus-lens moving frame 36b is moved in the same direction, that is, the direction along the optical axis O1 (arrow Y1 direction). The moving amount and direction of the focus lens 36a in this case can be set by the rotating amount and direction of the focus dial 36d. Therefore, a user (operator) can focus an observation image or an endoscopic image by the image pickup means by arbitrarily operating the focus dial 36d.

The zoom mechanism 37 mainly comprises a first-group zoom lens 37a constituting a part of a zoom optical system, a second-group zoom lens 37b similarly constituting a part of the zoom optical system, a zoom-lens moving frame 37c for fixing/holding the second-group zoom lens 37b, a guide pin 37d constituting a part of a zoom lens moving mechanism, and a zoom dial 37e having a guide groove 37ea for guiding movement of the guide pin 37d.

The first-group zoom lens 37a constituting a part of the zoom optical system is located on the optical axis O1 and installed securely at a fixing portion inside the camera adapter 12C.

The second-zoom lens 37b similarly constituting a part of the zoom optical system is located on the optical axis O1 and disposed movably in the direction along the optical axis O1 for focusing.

The zoom-lens moving frame 37c is disposed movably in the direction along the optical axis O1 in an internal space of the camera adapter 12C, while fixing/holding the second-group zoom lens 37b.

The guide pin 37d is made of a shaft-state member embedded outward with respect to the zoom-lens moving frame 37c and engaged with a cam groove 12k formed on the peripheral surface of the body portion of the camera adapter 12C. That is, the guide pin 37d and the cam groove 12k constitute the zoom-lens moving mechanism for moving the second-group zoom lens 37b in the direction along the optical axis O1 (arrow Y2 direction in FIG. 7).

The zoom dial 37e is arranged rotatably along the outer circumferential surface of the body portion of the camera adapter 12C. On the inner side of the zoom dial 37e, the guide groove 37ea is formed. At this guide groove 37ea, the tip end portion of the above-mentioned guide pin 37d is held.

By this configuration, when the operator rotates the zoom dial 37e in the direction along the outer circumferential surface of the body portion of the camera adapter 12C, with this rotation, the zoom dial 37e guides and rotates the guide pin 37d in the same direction through the guide groove 37ea.

Since the guide pin 37d is engaged with the cam groove 12k of the body portion of the camera adapter 12C, it is moved along the cam groove 12k. By this movement, the guide pin 37d moves the zoom-lens moving frame 37c in an arrow Y2 direction in FIG. 7, that is, the direction along the optical axis O1.

Each of the above optical systems arranged in the camera adapter 12C, that is, the focus lens 36a, the first-group zoom lens 37a, the second-group zoom lens 37b and the image forming lens 12d, is arranged on the same axis, that is, on the optical axis O1.

In the state where the camera adapter 12C and the illumination adapter 18C, which will be described later, are connected (state in FIG. 7), the optical axis O1 of each of the optical systems (36a, 37a, 37b, 12d) on the camera adapter 12C side and the center axis of the through hole 18c of the illumination adapter 18C are constituted to substantially match each other.

In the state where the camera adapter 12C and the endoscope 13 are connected (state in FIG. 9), the optical axis of each of the optical systems (36a, 37a, 37b, 12d) on the camera adapter 12C side and the optical axis of the ocular lend 13c of the endoscope 13 are constituted to substantially match each other on the same axis, that is, on the optical axis O1.

The optical axis O1 is a direction substantially orthogonal to the light receiving surface of the image pickup device 11a and is set to pass through substantially the center part of the light receiving surface of the image pickup device 11a, which is the same as the above first embodiment.

The other configurations, that is, the connecting means or the like of the camera adapter 12C and the camera head 11 as well as the illumination adapter 18C and the endoscope 13 is the same as that of the above first embodiment.

Next, the illumination adapter 18C constituting a part of the camera unit comprises an illumination position adjusting mechanism 35 made of a movable mirror or the like, which is illumination light reflecting means in place of the prism 18f in the above first embodiment as shown in FIG. 7.

This illumination position adjusting mechanism 35 is provided inside the projecting portion 18b of the illumination adapter 18C and is an illumination optical path changing optical system for ejecting an illumination light in a desired direction in a predetermined range by arbitrarily changing the ejecting angle so as to enable adjustment of the illumination direction at ejection of the illumination light from the illumination light ejection portion 14a of the light guide 14 connected to the illumination adapter 18C to the ejecting opening 18g through change of the optical path of the illumination light. The detailed configuration is as follows.

That is, the illumination position adjusting mechanism 35 mainly comprises a reflective mirror 35a for deflecting the optical axis O2 of the illumination light (See FIG. 7) so as to change the optical path of the illumination light by receiving the illumination light from the light guide 14 and reflecting it to a predetermined direction, a mirror moving frame 35b for fixing/holding this reflective mirror 35a, a mirror rotating shaft 35c for pivotally supporting the mirror moving frame 35b rotatably, and an illumination position adjusting lever 35d provided consecutively on the back face side of the mirror moving frame 35b for arbitrarily rotating the mirror moving frame 35b.

The illumination position adjusting mechanism 35 is disposed in the internal space of the projecting portion 18b of the illumination adapter 18C formed at a portion opposite to the illumination light ejection portion 14a, that is, on the optical path of the illumination light ejected from the illumination light ejection portion 14a in the state where the light guide 14 is connected to the illumination adapter 18C (state in FIG. 7).

In this case, the mirror rotating shaft 35c is pivotally supported by the internal fixing member (not shown particularly) of the illumination adapter 18C along the direction orthogonal to the optical axis O2 of the illumination light ejected from the illumination light ejection portion 14a.

At this mirror rotating shaft 35c, the mirror moving frame 35b is rotatably disposed with the mirror rotating shaft 35c as its rotating center.

On one face of this mirror moving frame 35b, the reflective mirror 35a is integrally fixed.

This reflective mirror 35a is disposed on the face of the mirror moving frame 35b so that its reflective surface is faced with the illumination light ejection portion 14a and the ejecting opening 18g, respectively, in the state shown in FIG. 7, that is, where the light guide 14 is connected to the illumination adapter 18C.

That is, the reflective mirror 35a is arranged so that its section is inclined toward the optical axis O2 of the illumination light ejected from the illumination light ejection portion 14a. By this, the illumination light from the illumination light ejection portion 14a is reflected by the reflective surface of the reflective mirror 35a toward the ejecting opening 18g.

In this case, the inclination angle of the reflective mirror 35a with respect to the optical axis O2 can be arbitrarily changed in a predetermined range by the illumination position adjusting lever 35d.

That is, at the mirror moving frame 35b, the illumination position adjusting lever 35d is integrally disposed so as to project outward from the back face. And the tip end portion of this illumination position adjusting lever 35d is exposed outside of the projecting portion 18b of the illumination adapter 18C. By this, the vicinity of the tip end portion of the illumination portion adjusting lever 35d acts as an operating member for position control so that the illumination light can illuminate a desired portion.

In this case, a hole (not shown particularly) through which the tip end of the illumination position adjusting lever 35d is penetrated to be exposed outside is formed at the projecting portion 18b of the illumination adapter 18C. This hole is formed in an elongated hole state having a predetermined length dimension so as to guide the illumination position adjusting lever 35d in the arrow R2 direction shown in FIG. 7 capable of inclination and to regulate its movable range.

The movable range of the illumination position adjusting lever 35d is regulated by the longitudinal direction of the elongated hole formed at the projecting portion 18b of the illumination adapter 18C as mentioned above. The longitudinal direction of this elongated hole also regulates the inclination angle of the reflective mirror 35a with respect to the optical axis O2. The movable range of the illumination position adjusting lever 35d is set within such a range that the inclination angle of the reflective mirror 35a is held when the illumination light after being ejected from the illumination light ejection portion 14a and reflected by the reflective surface of the reflective mirror 35a can be surely ejected from the ejecting opening 18g.

The other configurations are totally the same as those of the above-mentioned first embodiment.

In the endoscope system of this embodiment configured as above, action when used in the camera unit form by connecting the image pickup means and the illumination adapter 18C is as follows.

First, the state in FIG. 7 shows a case where the observation portion (operated portion) A2 located at a portion having a distance (working distance. Observation distance) shown by reference character WD1 from the camera unit is to be observed.

For observation in this state, first, the operator fixed the position of the camera unit so that an image of a predetermined region can be captured with the desired observation portion (operated portion) A2 at the center.

When the position of the camera unit is set, an incident angle E1 (See FIG. 7) with respect to the surface of the observation portion (operated portion) A2 of the optical axis O1 is set using the illumination position adjusting mechanism 35 so that the desired image pickup region is properly illuminated by the illumination light, that is, the optical axis O1 of the image pickup means (observation system) and the optical axis O2 of the illumination light cross each other at the observation portion (operated portion) A2.

That is, the operator holds the vicinity of the tip end portion of the illumination position adjusting lever 35d with the fingers and inclines it in the direction along the arrow R2 in FIG. 7. Then, following that, the mirror moving frame 35b is rotated in the direction inclined with respect to the optical axis O2 with the mirror rotating shaft 35c as its rotating center.

At this time, the illumination light from the illumination light ejection portion 14a is reflected by the reflective surface of the reflective mirror 35a and then, ejected toward the ejecting opening 18g. The inclined amount of the illumination position adjusting lever 35d is adjusted so that the optical axis O2 of this illumination light is in the state shown in FIG. 7, that is, ejected toward the desired observation portion (operated portion) A2.

After adjustment has been complete so that the desired image pickup region can be properly illuminated by the illumination light, then, the operator sets a desired image pickup magnification by rotating the zoom dial 37e in a predetermined direction as appropriate. Also, by rotating the focus dial 36d in a predetermined direction as appropriate, focus control operation is carried out. In this case, the operator performs various operations of variable power and focusing while looking at an observation image displayed on the monitor 24.

Action at setting change of the camera unit from the state shown in FIG. 7 to the state shown in FIG. 8 will be described. The state shown in FIG. 8 shows a case of observation of the observation portion (operated portion) A2 at a portion with a distance shown by the reference character WD2 from the camera unit. Here, the distance WD2 is exemplified as a case shorter than the distance WD1 shown in FIG. 7 (WD1>WD2).

First, the operator operates the arm lock/unlock switch 15b to unlock the holder portion 15 and then, the camera unit in the state in FIG. 7 (distance WD1) is moved to the state in FIG. 8 (distance WD2). Then, by operating the arm lock/unlock switch 15b at the position so as to lock the holder portion 15, the camera unit is fixed at a predetermined position in the space.

Then, according to the same procedure as mentioned above, the axial control of the illumination light, variable power setting and focusing setting are performed. Here, the axial control of the illumination light is performed as follows.

In the state shown in FIG. 8, the distance WD2 is set shorter than the distance WD1 shown in FIG. 7. Thus, the incident angle E2 of the optical axis O2 of the illumination light with respect to the observation portion (operated portion) A2 becomes smaller than the above-mentioned state in FIG. 7 (E1>E2).

Therefore, the illumination position control lever 35d is inclined from the state in FIG. 7 in the direction along an arrow R2a shown in FIG. 8. By this, the reflective mirror 35a is rotated clockwise in FIG. 8 around the mirror rotating shaft 35c. And the control is made so that the optical axis O1 of the image pickup means and the optical axis O2 of the illumination light cross each other at the observation portion (operated portion) A2.

On the other hand in the endoscope system of this embodiment, action that the illumination adapter 18C is removed from the camera unit form shown in FIGS. 7 and 8 and the light guide 14 is removed from this illumination adapter 18C or action that the endoscope 13 is mounted to the image pickup means and the endoscope 13 is connected to the light guide 14 so as to constitute the endoscope unit form (See FIG. 9) is performed with totally the same procedure as that of the above-mentioned first embodiment.

As mentioned above, according to the fourth embodiment, by arranging the reflective mirror 35a rotatably in the direction inclined toward the optical axis O2 of the illumination light, the optical path of the illumination light can be set arbitrarily, and the irradiated position of the illumination light can be set at a desired portion. Thus, when used in the camera unit form, even if the distance WD between the camera unit and the operated portion A2 to be the observation portion is changed, control can be made so that the optical axis O2 of the illumination light can be matched to the intersection between the optical axis O1 of the image pickup means and the desired observation portion (operated portion) A2 all the time. Therefore, regardless of the distance WD between the camera unit and the observation portion (operated portion), efficient illumination can be applied all the time to the desired observation portion.

Also, arrangement of both the dials 36d and 37e is made so that the surface including the rotation operating direction of the focus dial 36d and the surface including the rotation operating direction of the zoom dial 37e are substantially orthogonal to each other.

That is, each of the operating directions of the zoom dial 37e and the focus dial 36d is made as different rotation operating directions, and the focus dial 36d requiring finer operation is configured so as to be rotated/operated on the same face as the one including the direction along the optical axis O1 of the image pickup means in this case. By this, display control of the observation image can be made in more detail, which contributes to improvement of operability.

### Fifth Embodiment

Next, an endoscope system of a fifth embodiment of the present invention will be described below.

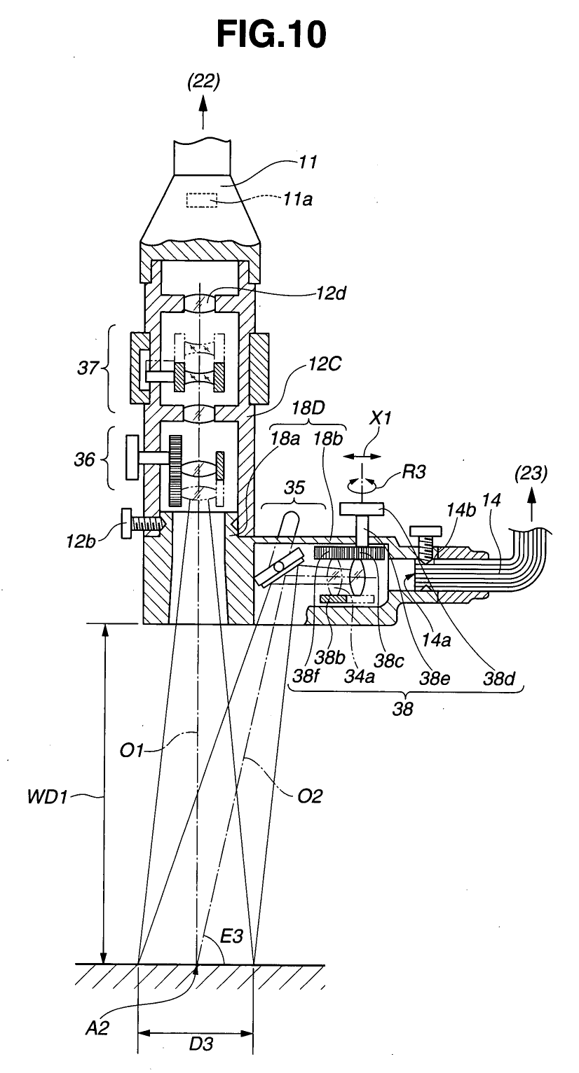

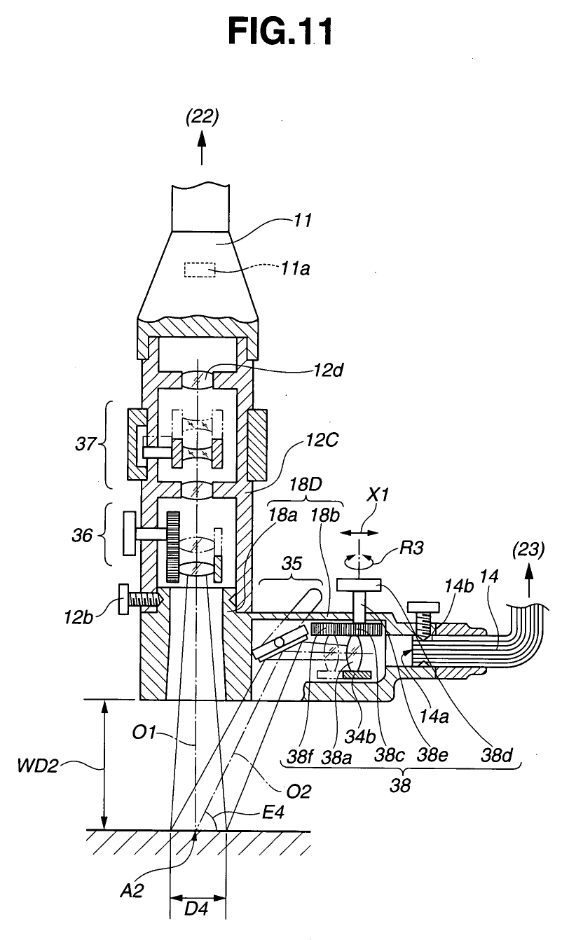

FIGS. 10 and 11 are longitudinal sectional views showing an outline configuration of a camera unit in the endoscope system of the fifth embodiment of the present invention. In them, FIG. 10 shows an example of the case where the distance (WD1) to the observation portion is long. FIG. 11 shows an example of the case where the distance (WD2) to the observation portion is shorter than the case in FIG. 10 (WD1).

The endoscope system of this embodiment is comprised by substantially the same configuration as that of the above-mentioned fourth embodiment. In addition, in this embodiment, the condenser lens 34a is also provided at an illumination adapter 18D as with the above third embodiment, and the illumination region adjusting mechanism 38 which enables to move the condenser lens 34a is provided so that the illumination region and the image pickup region can substantially match each other. Therefore, the same configuration as that of the fourth embodiment will be given the same reference numeral and the detailed description will be omitted, but only the difference will be described below.

The image pickup region by the image pickup means is changed according to the distance between the camera unit and the observation portion and the observation power of the image pickup means. Then, it is preferable that the illumination region of the illumination light from the light guide 14 ejected to the desired observation portion through the illumination adapter 18D substantially matches the image pickup region by the image pickup means.

The illumination adapter 18D in the endoscope system of this embodiment comprises the illumination region adjusting mechanism 38 for controlling the illumination region of the illumination light by operating the condenser lens 34a in the direction along the optical axis O2 of the illumination light.

The illumination region adjusting mechanism 38 mainly comprises the condenser lens 34a arranged at a position opposed to the illumination light ejection portion 14a of the light guide 14 and disposed movably in the direction along the optical axis O2 of the illumination light from the light guide 14 (direction along an arrow X1 in FIG. 10) for adjusting the condensing region, a condenser lens moving frame 38b provided with a rack 38f constituting a part of the condenser lens moving mechanism for moving the condenser lens 34a in the direction along the optical axis O2 for fixing/holding the condenser lens 38a, a condensing region adjusting gear 38c meshed with the rack 38f of the condenser lens moving frame 38b and constituting a part of the condenser lens moving mechanism, a condensing region adjusting dial rotating shaft 38e pivotally supporting the condensing region adjusting gear 38c capable of rotation, and a condensing region adjusting dial 38d provided on the axis on the other end side of the condensing region adjusting dial rotating shaft 38e and disposed outside the projecting portion 18b of the illumination adapter 18D.

By this configuration, when the condensing region adjusting dial 38d is rotated in an arrow R3 direction in FIG. 10, that is, around the condensing region adjusting dial rotating shaft 38e, the condensing region adjusting gear 38c is rotated in the same direction with that. Then, the rotating force of the condensing region adjusting gear 38c is transmitted to the rack 38f meshed with the condensing region adjusting gear 38c. By this, the rack 38f moves the condenser lens moving frame 38b in the direction along the arrow X1 shown in FIG. 10, that is, the direction along the optical axis O2. With this movement, the condenser lens 34a fixed/held by the condenser lens moving frame 38b is moved in the same direction, that is, the direction along the optical axis O2 (arrow X1 direction). The moving amount and direction of the condenser lens 34a in this case can be set by the rotating amount and direction of the condensing region adjusting dial 38d. Therefore, by arbitrarily operating the condensing region adjusting dial 38d, such control can be made that the image pickup region by the image pickup means and the illumination region of the illumination light by the light guide 14 are substantially matched to each other.

The other configuration is totally the same as that of the above-mentioned fourth embodiment.

In the endoscope system of this embodiment configured as above, action when the image pickup means and the illumination adapter 18D are connected to be used in the camera unit form is as follows.

First, the state in FIG. 10 shows a case where the observation portion (operated portion) A2 at a portion with the distance shown by reference character WD1 (working distance. Observation distance) from the camera unit is to be observed.

For observation in this state, first, the operator fixes the position of the camera unit so that an image of a predetermined region around the desired observation portion (operated portion) A2 can be captured. And by the same procedure as that of the above-mentioned fourth embodiment, the optical axis O1 of the image pickup means and the optical axis O2 of the illumination light are set to be substantially matched using setting of a desired image pickup magnification by rotating operation of the zoom dial 37e, focusing operation by the rotating operation of the focus dial 36d and the illumination position adjusting mechanism 35.

Then, using the illumination region adjusting mechanism 38, the illumination region is controlled so that the image pickup region and the illumination region substantially match each other so that a desired image pickup region can be properly illuminated.

That is, the operator rotates the condensing region adjusting dial 38d in the direction along the arrow R3 in FIG. 10. Then, following this, the condensing region adjusting gear 38c is rotated in the same direction. The rotating force of the condensing region adjusting gear 38c is transmitted to the rack 38f, by which the condenser lens moving frame 38b and the condenser lens 34a are moved in the direction along the arrow X1 shown in FIG. 10 (direction along the optical axis O2).

The operator sets the moving amount and direction of the condenser lens 34a by arbitrary rotating operation of the rotating amount and direction of the condensing region adjusting dial 38d, by which the desired illumination region D3 (See FIG. 10) is set.

In this way, the user (operator) makes control so that the image pickup region by the image pickup means and the illumination region of the illumination light by the light guide 14 are substantially matched as reference character D3 shown in FIG. 10 by arbitrarily operating the condensing region adjusting dial 38d.

These setting operations are made while looking at the observation images displayed on the monitor 24.

Next, action when the setting of the camera unit is changed from the state shown in FIG. 10 to the state shown in FIG. 11 will be described. The state shown in FIG. 10 shows a case where the observation portion (operated portion) A2 at a portion having the distance shown by the reference character WD2 from the camera unit is to be observed. Here, the distance WD2 exemplifies a case where it is shorter than the distance WD1 shown in the above FIG. 10 (WD1>WD2).

First, the operator operates the arm lock/unlock switch 15b to unlock the holder portion 15 and then, moves the camera unit in the state in FIG. 10 (distance WD1) to the state in FIG. 11 (distance WD2). Then, by operating the arm lock/unlock switch 15b at that position so as to lock the holder portion 15 and the camera unit is fixed at a predetermined position in the space.

Then, with the same procedure as mentioned above, various settings as axial control of the illumination light, control of the illumination region, setting of variable power, setting of focusing and the like are made. The control of the illumination region in this case is such that the image pickup region and the illumination region are substantially matched with each other as reference character D4 shown in FIG. 11.

On the other hand, in the endoscope system of the embodiment, an action that the illumination adapter 18D is removed from the camera unit form shown in FIGS. 10 and 11 and the light guide 14 is removed from this illumination adapter 18D or that the endoscope 13 is mounted to the image pickup means and the light guide 14 is connected to the endoscope 13 so as to configure the endoscope unit form (not shown particularly. See FIG. 9) is carried out with totally the same procedure as that of the above first embodiment.

As mentioned above, according to the fifth embodiment, the same effect as that of the fourth embodiment can be obtained. In addition, in this embodiment, the condenser lens 34a is provided at the illumination adapter 18D and the illumination region adjusting mechanism 38 for making the condenser lens 34a movable in the direction along the optical axis O2 is provided. By this, when used in the camera unit form, control can be made such that the image pickup region by the image pickup means and the illumination region including the desired observation portion (operated portion) A2 substantially match each other all the time even if the distance WD between the camera unit and the operated portion A2 to be the observation portion is changed or the image pickup power is changed. Therefore, regardless of the distance WD between the camera unit and the observation portion (operated portion) or image pickup magnification, efficient illumination can be performed all the time to the desired image pickup portion including the desired observation portion.

In each of the above-mentioned embodiments, the light guide 14 is configured so that it is detachably attached to each of the illumination adapter (18, 18A, 18B, 18C, 18D) and the endoscope (13). When the image pickup means is connected to the illumination adapter (18, 18A, 18B, 18C, 18D) to be used in the camera unit form, the light guide 14 is connected to the illumination adapter (18, 18A, 18B, 18C, 18D). Alternatively, when the image pickup means is connected to the endoscope (13) to be used as the endoscope unit, the light guide 14 is connected to the endoscope (13).

On the other hand, it can be considered to configure the light guide 14 and the camera adapter (12, 12A, 12C) integrally. In this configuration, when switching is to be made between the camera unit form and the endoscope unit form, it is only necessary to attach/detach only the illumination adapter and the endoscope, which is very convenient. This case will be described below as a sixth embodiment of the present invention.

### Sixth Embodiment

Next, an endoscope system of a sixth embodiment of the present invention will be described below.

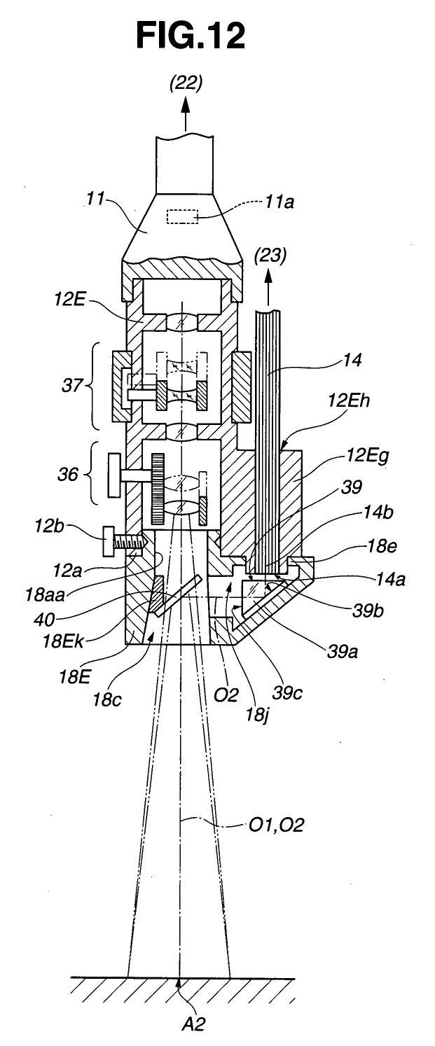

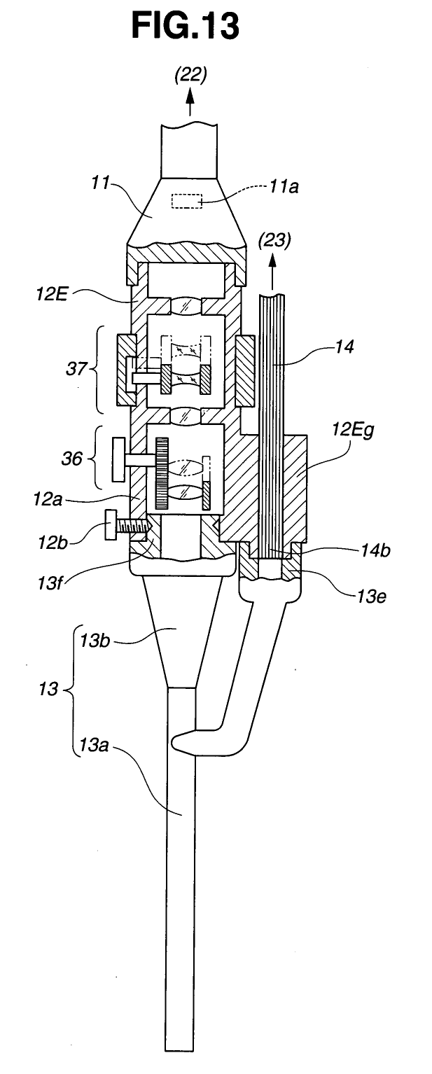

FIGS. 12 and 13 show the sixth embodiment of the present invention, in which FIG. 12 is a longitudinal sectional view showing an outline configuration of the camera unit in the endoscope system of this embodiment. FIG. 13 is a longitudinal sectional view showing an outline configuration of the endoscope unit in the endoscope system of this embodiment.

The endoscope system of this embodiment basically has the same configuration as the above-mentioned first embodiment and the fourth embodiment. This embodiment is different from them in the point, as mentioned above, that the light guide 14 and a camera adapter 12E are configured integrally, and an illumination adapter 18E having the internal configuration corresponding to it is provided. Therefore, the same configuration as those of the above-mentioned first and the fourth embodiments is given the same reference numeral and the detailed description will be omitted, and only the difference will be described below.

A camera adapter 12E in the image pickup means constituting the camera unit in this embodiment has, as shown in FIG. 12, a light-guide holding portion 12Eg projected in the vicinity of the outer circumferential edge portion on the tip end side of its body portion. Inside the light-guide holding portion 12Eg, a through passage 12Eh is formed in the direction along the optical axis O1 passing through the body portion of the camera adapter 12E. In this through passage 12Eh, the light guide 14 is inserted.

At the tip end side of the light guide 14, the second mounting portion 14b (second connection portion) to be a connection portion between the second adapter connection portion 18e of the illumination adapter 18E and a connection portion with the LG connection portion 13e of the endoscope 13 is formed. The tip end face of this second mounting portion 14b is the illumination light ejection portion 14a from which the illumination light is ejected.

At the illumination adapter 18E, two connection portions to be engaged at connection to the camera adapter 12E are formed, that is, a connection portion 18aa corresponding to the first mounting portion 12a (first connection portion) on the camera adapter 12E side and a second adapter connection portion 18e corresponding to the second mounting portion 14b (second connection portion) on the camera adapter 12E side.

Inside the connection portion 18aa of these two connection portions (18aa, 18e), the through hole 18c is formed. And when the illumination adapter 18E is connected to the camera adapter 12E, the through hole 18c is consecutively installed in the optical path of the camera adapter 12E and forms the optical path with the optical axis O1 of the image pickup means.

Inside the second adapter connection portion 18e, an optical path 18j is formed through which the illumination light from the light guide 14 connected to that is passed, and the optical path 18j communicates with the above through hole 18c.

Inside the illumination adapter 18E, a triangular prism 39 made of an optical prism and the like for deflecting the optical axis O2 of the illumination light from the light guide 14 and a half mirror 40 made of a semi-transmissive mirror or the like reflecting the illumination light form the triangular prism 39 to the observation portion are disposed at the respective predetermined portions.

Among them, the triangular prism 39 is disposed on the optical path 18j inside the illumination adapter 18E. This triangular prism 39 is disposed at a position opposite to the illumination light ejection portion 14a of the light guide 14 on the camera adapter 12E side when the illumination adapter 18E and the camera adapter 12E are connected to each other. And this triangular prism 39 comprises an incident surface 39a arranged opposite to the illumination light ejection portion 14a and into which the illumination light from the illumination light ejection portion 14a is entered, a reflective surface 39b formed with an inclination angle of approximately 45 degrees with respect to this incident surface 39a for reflecting the illumination luminous flux incident from the incident surface 39a, and an ejecting surface 39c formed with an inclination angle of approximately 45 degrees with respect to this reflective surface 39b for ejecting the illumination light reflected by the reflective surface 39b.

By this configuration, the illumination light ejected from the illumination light ejection portion 14a toward the triangular prism 39 enters from the incident surface 39a and is reflected by the reflective surface 39b so that the optical axis O2 is deflected by an angle of approximately 90 degrees to eject from the ejecting surface 39c.

Also, the half mirror 40 is disposed at a position opposite to the ejecting surface 39c of the triangular prism 39 on the optical axis O2 of the illumination light and on the optical axis O1 of the image pickup means. This half mirror 40 is fixed by a fixing portion 18Ek inside the illumination adapter 18E.

In this case, the half mirror 40 is arranged with an inclination angle of approximately 45 degrees with respect to the optical axis O1 of the image pickup means. And the reflective surface of the half mirror 40 is arranged facing the observation portion side. At the same time, the reflective surface of the half mirror 40 is arranged with an inclination angle of approximately 45 degrees also with respect to the optical axis O2 of the illumination light. And the reflective surface of the half mirror 40 is arranged facing the ejecting surface 39c of the triangular prism 39.

Therefore, by this configuration, the half mirror 40 receives the illumination light ejected from the ejecting surface 39c of the triangular prism 39 and reflects it toward the observation portion side. At the same time, the half mirror 40 transmits the luminous flux from the observation portion (luminous flux reflected by the observation object at the observation portion and enters the camera unit side) so that the image pickup luminous flux toward the (image pickup device 1 a of the) image pickup means of the camera unit is not disturbed.

On the other hand, as shown in FIG. 13, when the endoscope 13 is connected to the camera adapter 12E in this embodiment to be used as the endoscope unit, the camera connection portion 13f of the endoscope 13 is connected to the first mounting portion 12a (first connection portion) of the camera adapter 12E, and the LG connection portion 13e of the endoscope 13 is also connected to the second mounting portion 14b (second connection portion) of the camera adapter 12E.

As the endoscope 13 applied in this embodiment, the same one as that of the above-mentioned first embodiment can be applied. Therefore, the detail of the configuration will be omitted.

Also, in this embodiment, by integrally constituting the light guide 14 and the camera adapter 12E, the illumination light from the light guide 14 is guided so as to overlap the optical axis O1 by the triangular prism 39 and the half mirror 40 fixed on the optical axis O2, and the illumination position adjusting mechanism 35 in the above-mentioned fourth embodiment is not needed any more.

The other configurations and actions are substantially the same as those of the above first embodiment or the fourth embodiment.

As mentioned above, according to the above sixth embodiment, the same effects as those of the first and the fourth embodiments can be obtained. In addition, according to this embodiment, by integrally constituting the light guide 14 and the camera adapter 12E, when switching is made between the camera unit form and the endoscope unit form, it is only necessary to attach/detach the illumination adapter 18E and the endoscope 13. Therefore, it contributes to improvement of operability.

Also, since the illumination light from the light guide 14 is guided coaxially with the optical axis O1 of the image pickup means, appropriate illumination can be given all the time. Also, even if the observation portion is in the deep hole shape, illumination can be given from the same direction as the image capturing direction, and a shadow is not formed but efficient illumination can be obtained.

In each of the above embodiments, the camera adapter (12, 12A, 12C, 12E) and the illumination adapter (18, 18A, 18B, 18C, 18D) are configured capable of attachment/detachment. By this, the illumination adapter (18, 18A, 18B, 18C, 18D) can be connected to the camera adapter (12, 12A, 12C, 12E) to have the camera unit form, while the illumination adapter (18, 18A, 18B, 18C, 18D) is removed from this camera unit and then, by mounting the endoscope (13) instead of this, the endoscope unit form can be configured.

On the other hand, it can be considered that the above camera adapter (12, 12A, 12C) and the illumination adapter (18, 18A, 18B, 18C, 18D) can be configured integrally to have a camera unit. With this configuration, if switching is to be made between the camera unit form and the endoscope unit form, it is only necessary to attach/detach the endoscope with respect to the camera unit, which is very convenient. This case will be described below as a seventh, an eighth and a ninth embodiments of the present invention.

### Seventh Embodiment

Next, an endoscope system of a seventh embodiment of the present invention will be described below.

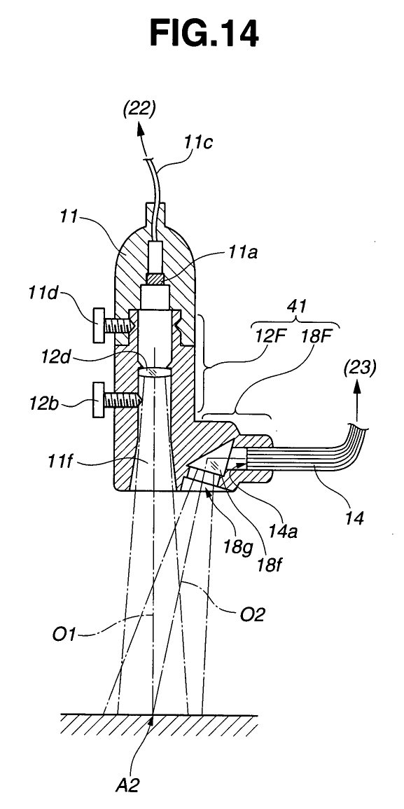

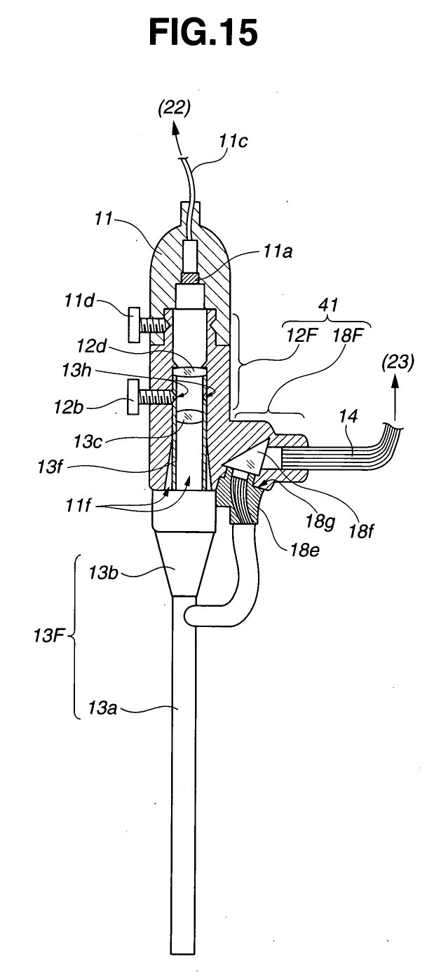

FIGS. 14, 15 show the seventh embodiment of the present invention, in which FIG. 14 is a longitudinal sectional view showing an outline configuration of a camera unit in an endoscope system of this embodiment. FIG. 15 is a longitudinal sectional view showing an outline configuration of the endoscope unit of the endoscope system of this embodiment.

The endoscope system of this embodiment basically has the same configuration as that of the above-mentioned first embodiment. This embodiment is different in the point that, as mentioned above, a camera illumination adapter 41 in which a body portion 12F corresponding to the camera adapter and an illumination holding portion 18F corresponding to the illumination adapter are integrally configured, and when used as the endoscope unit, the endoscope 13F is connected to the camera illumination adapter 41. Therefore, the same configuration as those of the above first and the fourth embodiments are given the same reference numerals and the detailed description will be omitted, but only the difference will be described below. Also, an inventive idea of this embodiment is substantially the same as that of the above sixth embodiment.

In this embodiment, the image pickup means comprises the camera head 11 having the image pickup device 11a and the camera illumination adapter 41 made of a body portion 12F and the illumination holding portion 18F. And the image pickup means in the form where the camera head 11 and the camera illumination adapter 41 are connected functions as the camera unit in each of the above embodiments in that state.

The camera illumination adapter 41 comprises, as shown in FIGS. 14, 15, the body portion 12F and the illumination holding portion 18F in the way that they have totally the same shape and configuration as the case where the camera adapter 12 and the illumination adapter 18 are connected in the above-mentioned first embodiment, and the both are integrally formed.

The body portion 12F is in the cylindrical shape having a through hole 11f inside and comprises the image forming lens 12d inside. The through hole 11f forms an optical path for transmitting the luminous flux from the observation portion incident from the opening on its tip end side. That is, the through hole 11f constitutes the optical path for having the luminous flux from the observation portion enter the image pickup means 11a when the camera illumination adapter 41 and the camera head 11 are connected to each other. The image forming lens 12d is fixed on the optical path of the through hole 11f. The image forming lens 12d transmits the luminous flux from the observation portion and has an optical image of the observation portion formed on the light receiving surface of the image pickup device 11a. Therefore, the optical axis O1 of the image pickup means passes through the through hole 11f.

The illumination holding portion 18F is projected outward from the vicinity of the outer circumferential edge portion of the body portion 12F. The illumination holding portion 18F functions as a light guide holding portion. Therefore, one end of the light guide 14 is connected and fixed at the end of the illumination holding portion 18F. Inside the illumination holding portion 18F, the prism 18f, which is an illumination optical path changing optical system is arranged.

The incident surface of this prism 18f is arranged at a portion opposite to the illumination light ejection portion 14a of the light guide 14. The ejecting surface of the prism 18f is arranged at the portion opposite to the ejecting opening 18g formed on the bottom face portion of the illumination holding portion 18F. And between the incident surface of the prism 18f and the illumination light ejection portion 14a of the light guide 14 and between the light ejecting surface of the prism 18f and the ejecting opening 18g of the illumination holding portion 18F, an illumination optical path is formed, respectively. Therefore, the illumination light from the light guide 14 is ejected from the illumination ejection portion 14a and then enters the incident surface of the prism 18f. The illumination light having entered the incident surface of the prism 18f has its optical axis O2 deflected by the reflective surface of the prism 18f and reflected to the ejecting surface of the prism 18f. The illumination light ejected to the ejecting surface of the prism 18f is ejected from the ejecting opening 18g of the illumination holding portion 18F to the outside to illuminate the observation portion.

On the other hand, in this embodiment, an endoscope 13F can be connected to the camera unit (image pickup means) in the form shown in FIG. 14.

That is, in this case, as shown in FIG. 15, the camera connection portion 13f of the base end 13b of the endoscope 13F is engaged with the opening on the tip end side of the through hole 11f of the illumination holding portion 18F of the camera illumination adapter 41. In this state, the first fixing member 12b is operated in the tightening direction. By this, the tip end of the first fixing member 12b is engaged with the engagement peripheral groove 13h of the camera connection portion 13f. The fixing procedure of the endoscope 13F by this first fixing member 12b is the same as the procedure of connecting and fixing the camera adapter 12 and the endoscope 13 to each other in the above-mentioned first embodiment.

Also, the LG connection portion 13e of the endoscope 13 is engaged and connected to the ejecting opening 18g of the illumination holding portion 18F. The endoscope unit is configured in this way.

Inside the camera connection portion 13f at the base end of the endoscope 13F, the ocular lens 13c is fixed. While the endoscope 13F is connected to the camera illumination adapter 41, the image pickup device 11a of the camera head 11, the image forming lens 12d on the camera illumination adapter 41 side, and the ocular lens 13c of the endoscope 13F are arranged on the same axis. By this, the endoscopic observation image formed by the ocular lens 13c of the endoscope 13F is formed again on the light receiving surface of the image pickup device 11a through the image forming lens 12d.

In this way, in this embodiment, the image pickup means in the state where the camera head 11 and the camera illumination adapter 41 are connected to each other can be used as the camera unit. On the other hand, by connecting a part (camera connection portion 13f) of the base end 13b of the endoscope 13F and the LG connection portion 13e to a predetermined portion (ejecting opening 18g of the illumination holding portion 18F) of the camera illumination adapter 41, the form is switched to the endoscope unit.

The other configurations and actions are substantially the same as those of the above-mentioned first or the fourth embodiments.

As mentioned above, according to the seventh embodiment, the same effect as that of the above-mentioned first embodiment can be obtained. In addition, in this embodiment, the camera illumination adapter 41 is provided in which the body portion 12F corresponding to the configuration of the camera adapter in each of the above embodiments and the illumination holding portion 18F corresponding to the illumination adapter in each of the above embodiments are integrally formed. Therefore, the image pickup means formed by connecting the camera illumination adapter 41 and the camera head 11 to each other can be used as a camera unit as it is.

Only by connecting and fixing the base end portion 13b of the endoscope 13F and the LG connection portion 13e to each of the predetermined portions of the camera adapter 41 in this camera unit (image pickup means), the form can be switched to the endoscope unit.

Moreover, in order to switch from this endoscope unit to the camera unit form, it is only necessary to remove the endoscope 13F (base end portion 13b and the LG connection portion 13e). Therefore, only by attaching/detaching the endoscope 13F to the camera unit, the form of each unit can be switched easily and surely, which can contribute to improvement of operability.

### Eighth Embodiment

Next, an endoscope system of an eighth embodiment of the present invention will be described below.

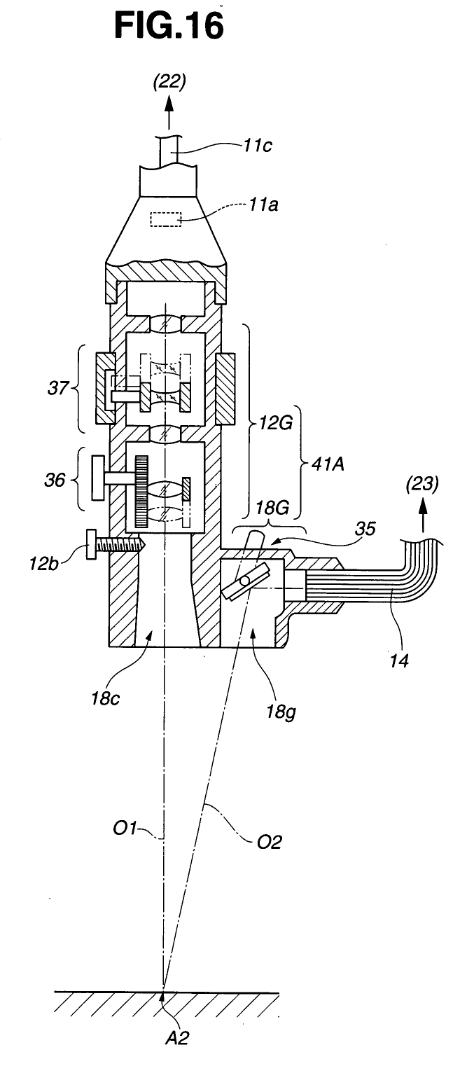

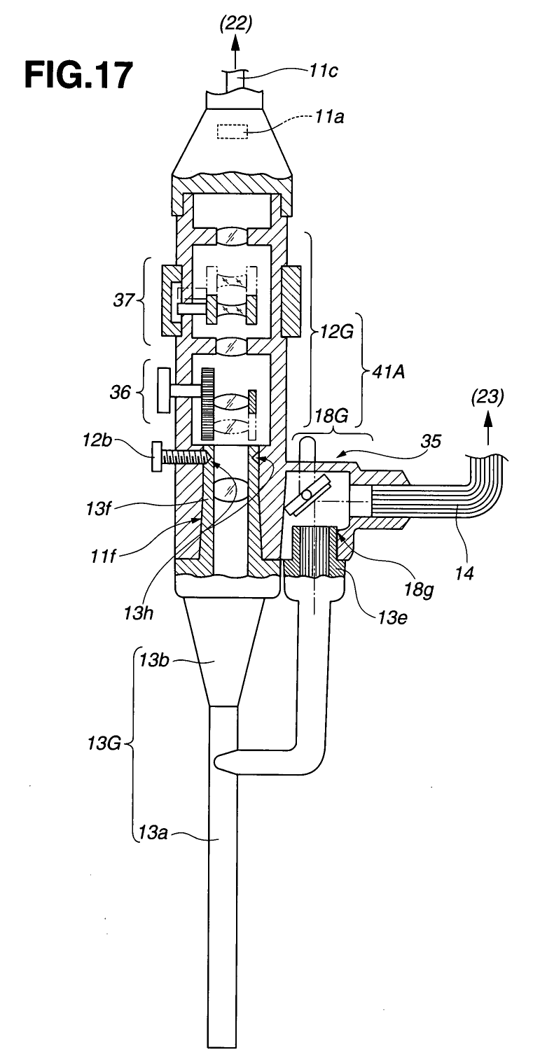

FIGS. 16, 17 shows the eighth embodiment of the present invention, in which FIG. 16 is a longitudinal sectional view showing an outline configuration of the camera unit in the endoscope system in this embodiment. FIG. 17 is a longitudinal sectional view showing an outline configuration of the endoscope unit in this endoscope system in this embodiment.

The endoscope system of this embodiment basically has the same configuration as that of the above-mentioned fourth embodiment. In this embodiment, a camera illumination adapter 41 A is provided as shown in FIG. 16, in which a body portion 12G corresponding to the camera adapter and an illumination holding portion 18G corresponding to the illumination adapter are configured integrally, and when used as the endoscope unit, an endoscope 13G is connected to the camera illumination adapter 41A as shown in FIG. 17.

An inventive idea of this embodiment is totally the same as that of the above-mentioned seventh embodiment. Therefore, the same configuration as that of the above first and the fourth embodiments is given the same reference numeral and the description will be omitted.

In this embodiment, the image pickup means is configured by the camera head 11 having the image pickup device 11a and the camera illumination adapter 41A made of the body portion 12G and the illumination holding portion 18G. And the image pickup means in the form that the camera head 11 and the camera illumination adapter 41A are connected functions as the camera unit as it is in each of the above embodiments.

In the camera illumination adapter 41 A, as shown in FIGS. 16, 17, the body portion 12G and the illumination holding portion 18G are configured respectively so that they are in totally the same shape and configuration as the case where the camera adapter 12C and the illumination adapter 18C are connected in the above-mentioned fourth embodiment and the both are integrally formed.

Here, the internal configuration of the body portion 12G is totally the same as that of the camera adapter 12C in the above-mentioned fourth embodiment, and the internal configuration of the illumination holding portion 18G is totally the same as that of the illumination adapter 18C in the above-mentioned fourth embodiment.

And to this camera illumination adapter 41A, the endoscope 13G can be connected using the first fixing member 12b as in the above-mentioned seventh embodiment (See FIG. 17).

In this case, the camera connection portion 13f of the base end portion 13b of the endoscope 13G is engaged with the tip-end side opening of the though hole 11f of the illumination holding portion 18G of the camera illumination adapter 41A. And by operating the first fixing member 12b in the tightening direction, the tip end of the first fixing member 12b is engaged with the engagement peripheral groove 13h of the camera connection portion 13f. By this, the endoscope 13G is fixed to the camera illumination adapter 41A.

Also, the LG connection portion 13e of the endoscope 13 is engaged and connected to the ejecting opening 18g of the illumination holding portion 18G. By this, the endoscope unit is configured.

The other configurations and actions are substantially the same as those of the above-mentioned first, fourth or seventh embodiment.

As mentioned above, according to the above eighth embodiment, the same effect as those of the above-mentioned first and the fourth embodiments can be obtained. In addition, in this embodiment, by applying the camera illumination adapter 41A in which the body portion 12G having the same shape and function as those of the camera adapter 12C in the above fourth embodiment and the illumination holding portion 18G having the same shape and function as those of the illumination adapter 18C in the fourth embodiment are integrally configured, the same effect as that in the above-mentioned seventh embodiment can be obtained.

### Ninth Embodiment

Next, an endoscope system of a ninth embodiment of the present invention will be described below.

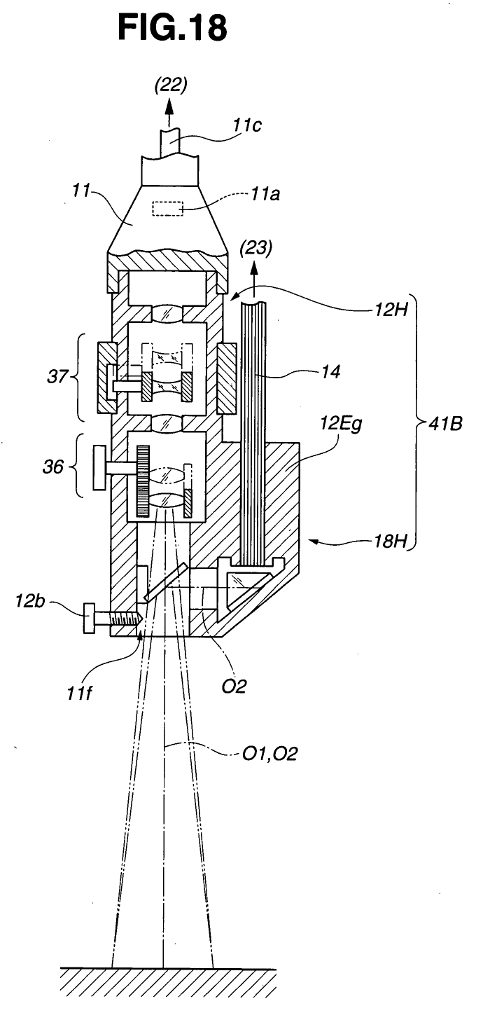

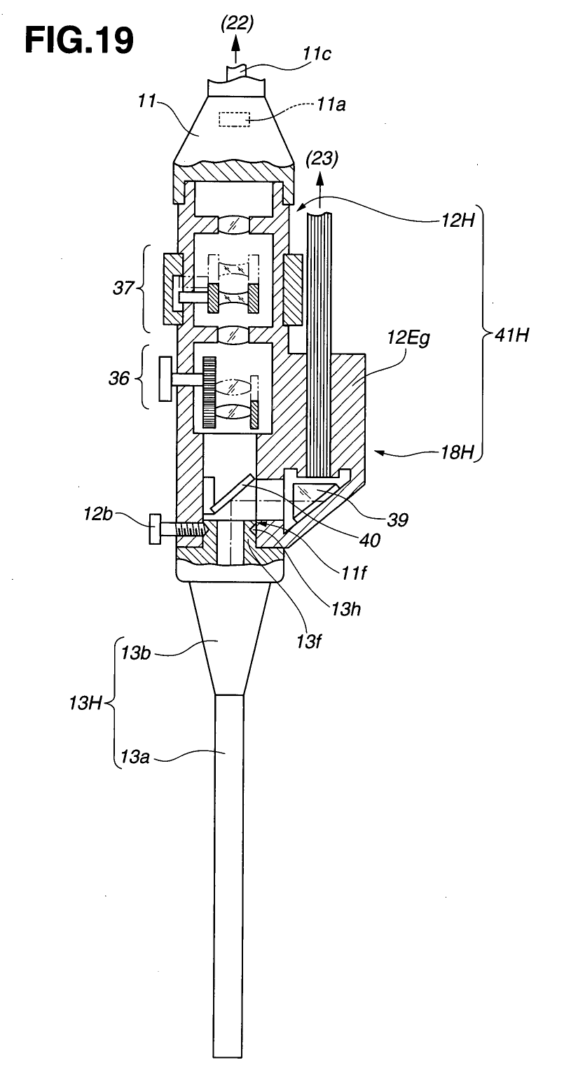

FIGS. 18 and 19 show a ninth embodiment of the present invention, in which FIG. 18 is a longitudinal sectional view showing an outline configuration of a camera unit in an endoscope system in this embodiment. FIG. 19 is a longitudinal sectional view showing an outline configuration of an endoscope unit of the endoscope system of this embodiment.

The endoscope system of this embodiment basically has the same configuration as that of the above-mentioned sixth embodiment. In this embodiment, as shown in FIG. 18, a camera illumination adapter 41H in which a body portion 12H corresponding to the camera adapter and an illumination holding portion 18H corresponding to the illumination adapter are integrally configured as shown in FIG. 18 is provided, and when used as the endoscope unit, the endoscope 13H is connected to the camera illumination adapter 41H as shown in FIG. 19.

The inventive idea of this embodiment is the same as that of the above-mentioned sixth, seventh embodiments. Therefore, the same configuration as that of the above first, fourth, sixth embodiment is given the same reference numeral and the description will be omitted.

In this embodiment, the image pickup means is comprised by the camera head 11 having the image pickup device 11a and the camera illumination adapter 41H made of the body portion 12H and the illumination holding portion 18H. And the image pickup means in the form that the camera head 11 and the camera illumination adapter 41H are connected functions as the camera unit as it is in each of the above embodiments.

In the camera illumination adapter 41H, as shown in FIGS. 18, 19, the body portion 12H and the illumination holding portion 18H are configured respectively so that they are in totally the same shape and configuration as the case where the camera adapter 12E and the illumination adapter 18E are connected in the above-mentioned sixth embodiment and the both are integrally formed.

Here, the internal configuration of the body portion 12H is totally the same as that of the camera adapter 12E in the above-mentioned sixth embodiment and also the same in the point that the light guide holding portion 12Eg is provided at the outer circumferential edge portion of the body portion 12H. Also, the internal configuration of the illumination holding portion 18H is totally the same as that of the illumination adapter 18C in the above-mentioned sixth embodiment.

To the camera illumination adapter 41H, the endoscope 13H can be connected as in the above-mentioned seventh, eighth embodiment using the first fixing member 12b (See FIG. 19).

In this case, the camera connection portion 13f of the base end portion 13b of the endoscope 13H is engaged with the tip-end side opening of the through hole 11f of the illumination holding portion 18H of the camera illumination adapter 41H. By operating the first fixing member 12b in the tightening direction in this state, the tip end of the first fixing member 12b is engaged with the engagement peripheral groove 13h of the camera connection portion 13f. By this, the endoscope 13H is fixed to the camera illumination adapter 41H.

In this embodiment, as with the above-mentioned sixth embodiment, the light guide holding portion 12Eg is integrally formed with the body portion 12H. That is, the light guide 14 is integrally disposed in the body portion 12H. By this, the attachment/detachment with respect to the light guide 14 is made unnecessary. Therefore, only by attaching/detaching the endoscope 13 with respect to the camera illumination adapter 41H, the state can be switched between the camera unit form and the endoscope unit form.

In correspondence with this, the endoscope 13H applied in this embodiment is a type in which the LG connection portion 13e is not provided. Therefore, in this embodiment, when the endoscope 13H is attached to the camera illumination adapter 41H, the illumination light from the light guide 14 has its optical axis O2 deflected by the triangular prism 39 and the half mirror 40 and guided to a position overlapping the optical axis O1 of the image pickup means. That is, in this embodiment, the illumination light from the light guide 14 illuminates the observation portion through the optical system for endoscopic image observation such as the relay optical system and the objective optical system of the endoscope 13H. Therefore, the configuration of the endoscope 13H applied in this embodiment does not have means such as light guide fiber bundles or the like for transmitting the illumination light.

The other configurations and actions are substantially the same as that of the above-mentioned first, fourth, sixth or seventh embodiment.

As mentioned above, according to the ninth embodiment, the same effect as that of the above-mentioned first, fourth, sixth embodiment can be obtained. In addition, with this embodiment, by applying the camera illumination adapter 41H in which the body portion 12H having the same shape and function as those of the camera adapter 12E in the above sixth embodiment and the illumination holding portion 18H having the same shape and function as those of the illumination adapter 18E in the sixth embodiment are integrally comprised, the same effect as those of the above-mentioned seventh, eighth embodiment can be obtained.

Moreover, the light guide 14 is integrally configured with the camera illumination adapter 41H. When used as the endoscope unit, the illumination light from the light guide 14 is transmitted to the observation portion through the optical system for observation of the endoscope 13H, and the endoscope 13H can be configured without the illumination light transmitting means.

### Tenth Embodiment

Next, an endoscope system of a tenth embodiment of the present invention will be described below.

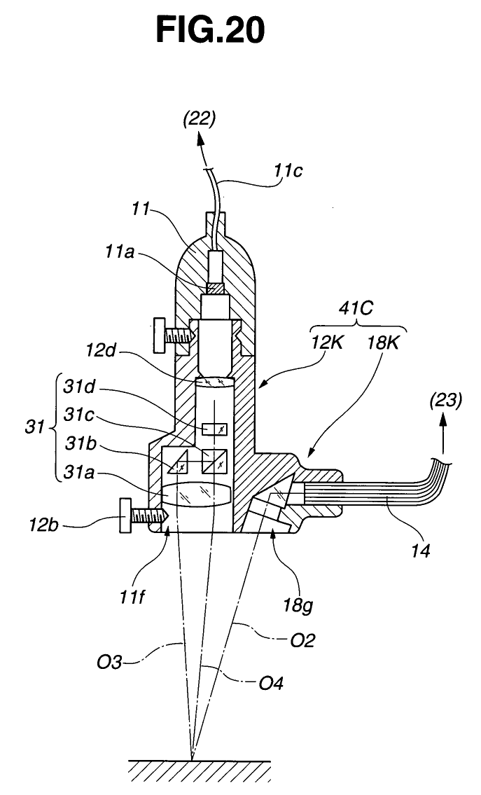

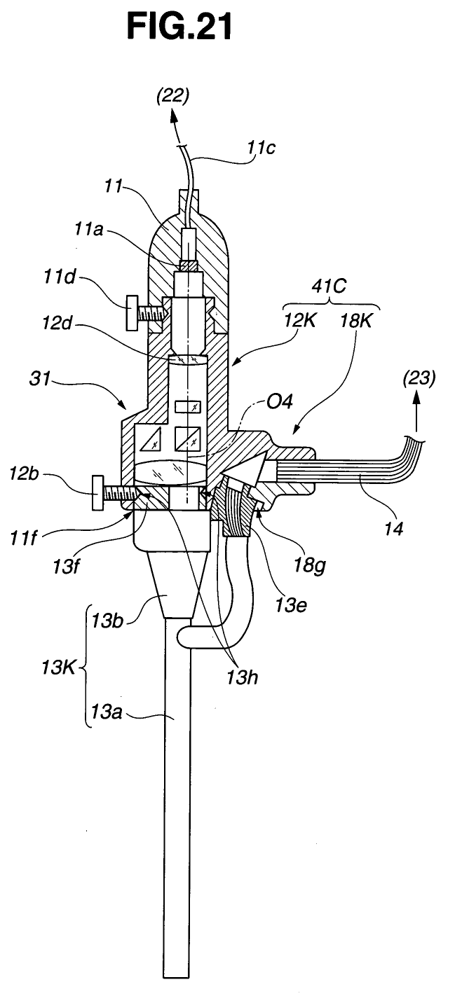

FIGS. 20, 21 show the tenth embodiment of the present invention, in which FIG. 20 is a longitudinal sectional view showing an outline configuration of the camera unit in an endoscope system of this embodiment. FIG. 21 is a longitudinal sectional view showing an outline configuration of the endoscope unit in the endoscope system of this embodiment.

The endoscope system of this embodiment basically has the same configuration as that of the above-mentioned second embodiment. In this embodiment, a camera illumination adapter 41 C in which a body portion 12K corresponding to the camera adapter and an illumination holding portion 18K corresponding to the illumination adapter are integrally configured as shown in FIG. 20 is provided, and when used as the endoscope unit, an endoscope 13K is connected to the camera illumination adapter 41 C as shown in FIG. 21.

The inventive idea of this embodiment is the same as that of the above-mentioned sixth, seventh, eighth embodiment. Therefore, the same configuration as that of the above-mentioned first and second embodiments is given the same reference numeral and the detailed description will be omitted.

In this embodiment, the image pickup means is configured by the camera head 11 having the image pickup device 11a and the camera illumination adapter 41 C made of the body portion 12K and the illumination holding portion 18K. And the image pickup means in the state that the camera head 11 and the camera illumination adapter 41C are connected to each other functions as the camera unit as it is in each of the above embodiments.

In the camera illumination adapter 41C, as shown in FIGS. 20, 21, the body portion 12K and the illumination holding portion 18K are configured respectively so that they are in totally the same shape and configuration as the case where the camera adapter 12A and the illumination adapter 18A are connected in the above-mentioned second embodiment and the both are integrally formed.

Here, the internal configuration of the body portion 12K is totally the same as that of the camera adapter 12A in the above-mentioned second embodiment. Also, the internal configuration of the illumination holding portion 18K is totally the same as that of the illumination adapter 18C in the above-mentioned second embodiment and is provided with the 3D observation optical system 31 and the prism 18f, which is the illumination optical path changing optical system as with the illumination adapter 18C in the above-mentioned second embodiment.

To the camera illumination adapter 41C, the endoscope 13K can be connected as in the above-mentioned seventh, eighth, ninth embodiment using the first fixing member 12b (See FIG. 21).

In this case, the camera connection portion 13f of the base end portion 13b of the endoscope 13K is engaged with the tip-end side opening of the through hole 1 I f of the illumination holding portion 18K of the camera illumination adapter 41C. By operating the first fixing member 12b in the tightening direction in this state, the tip end of the first fixing member 12b is engaged with the engagement peripheral groove 13h of the camera connection portion 13f. By this, the endoscope 13K is fixed to the camera illumination adapter 41C.

Also, in order to guide the illumination light of the light guide 14 disposed integrally at the camera illumination adapter 41 C, the LG connection portion 13 e of the endoscope 13K is connected to the ejecting opening 18g of the illumination holding portion 18K.

When used as the endoscope unit, only the observation image for the left eye is guided to the image pickup means 11a. Therefore, the endoscopic observation image handles an ordinary 2D image.

The other configurations and actions are substantially the same as those of the above-mentioned second or seventh, eighth embodiment and the like.

As mentioned above, according to the above tenth embodiment, the same effect of that of the above second embodiment can be obtained, that is, the 3D observation can be made. In addition, in this embodiment, by applying the camera illumination adapter 41C in which the body portion 12K having the same shape and function as those of the camera adapter 12A in the above-mentioned second embodiment and the illumination holding portion 18K having the same shape and function as those of the illumination adapter 18A in the second embodiment are integrally configured, the same effect as that of the above-mentioned seventh, eighth and ninth embodiment can be obtained.

In each of the above embodiments, there can be such a configuration that identifying means or state detecting means for identifying which of the endoscope (13) or the illumination adapter (18) is attached to the camera adapter (12), for example, is provided in the vicinity of the connection portion between the both. In this case, the detection result is transmitted to the CCU 22 through the cable 11c or the like and displayed, for example, so that the result can be checked on the monitor 24.

By visually checking the monitor 24 by the operator in this configuration, the operator can recognize easily and rapidly the state of the endoscope system. Therefore, it can greatly contribute to improvement of the operability.

In the present invention, it is apparent that different embodiments in a wide range can be configured based on the present invention without departing from the spirit and scope of the invention. The present invention is not restricted by particular embodiments except being limited by the appended claims.

What is claimed is:

1. An endoscope system comprising:
image pickup means having an image pickup device;
illumination light ejecting means having an illumination light ejection portion;
an endoscope having an insertion portion and having an illumination light incident portion for guiding illumination light to a base end of the insertion portion and an optical image ejection portion for ejecting light from a subject guided through the insertion portion;
a first connection portion provided at the image pickup means for attaching the endoscope to the image pickup means so that the light from the subject ejected by the optical image ejection portion can be captured by the image pickup device;
a second connection portion provided at the illumination light ejecting means for attaching the endoscope to the illumination light ejecting means so that the illumination light ejected by the illumination light ejection portion can enter the illumination light incident portion;
an adapter having a first adapter connection portion which can be attached to the first connection portion instead of the endoscope and a second adapter connection portion which can be attached to the second connection portion instead of the endoscope;
an optical path provided at the adapter for capturing an image of the subject by the image pickup device; and
an illumination optical system provided at the adapter for guiding the illumination light ejected from the illumination light ejection portion within an image pickup region for image pickup through the optical path.

2. The endoscope system according to claim 1, further comprising a holder portion for holding the image pickup device and the illumination light ejection portion.

3. The endoscope system according to claim 1, wherein the image pickup means and the illumination light ejecting means are configured integrally.

4. The endoscope system according to claim 1, wherein the optical axis of the illumination optical system overlaps the optical axis of the optical path coaxially.

5. The endoscope system according to claim 1, wherein the optical axis of the illumination optical system is set so as to cross the optical axis of the optical path at a predetermined portion.

6. The endoscope system according to claim 5, wherein the predetermined portion is an observation portion on the subject to be captured by the image pickup device.

7. The endoscope system according to claim 1, wherein the illumination optical system has an illumination light reflecting means for guiding the illumination light ejected by the illumination light ejection portion into the image pickup region of the image pickup means.

8. The endoscope system according to claim 1, wherein the adapter has an objective optical system, polarizing means for polarizing at least two different luminous fluxes from the objective optical system in a different direction, an optical system for guiding the at least two different luminous fluxes into the same optical path, and polarized luminous flux transmitting means for alternately transmitting at least two luminous fluxes with different polarized states.

9. The endoscope system according to claim 1, wherein identifying means is provided for identifying that the image pickup means and the adapter are in the connected state.

10. The endoscope system according to claim 1, wherein the illumination optical system has condensing means for narrowing an illumination range.

11. The endoscope system according to claim 7, wherein the illumination light reflecting means can change the optical path of the illumination light guided into the image pickup range of the image pickup means.

12. The endoscope system according to claim 10, wherein the condensing means can change the illumination range of the illumination light.

13. An endoscope system comprising:
an endoscope having an insertion portion and having an illumination light incident portion for guiding illumination light to a base end of the insertion portion and an optical image ejection portion for ejecting a light from a subject guided through the insertion portion; and
image pickup means having an image pickup device, an illumination light ejecting means having an illumination light ejection portion, a first connection portion for attaching the endoscope to the image pickup means so that the light from the subject ejected by the optical image ejection portion can be captured by the image pickup device, and a second connection portion provided at the illumination light ejecting means for attaching the endoscope to the illumination light ejecting means so that the illumination light ejected by the illumination light ejection portion can enter the illumination light incident portion; and
wherein the image pickup means has an illumination optical system for guiding the illumination light from the illumination light ejection portion to the illumination light incident portion when the endoscope is connected and for irradiating the illumination light from the illumination light ejection portion into an image pickup range captured by the image pickup device when the endoscope is separated.

14. The endoscope system according to claim 13, further comprising a holder portion for holding the image pickup device.

15. The endoscope system according to claim 13, wherein the optical axis of the illumination optical system is coaxial with the optical axis of the image pickup system for capturing the subject when the endoscope is separated.

16. The endoscope system according to claim 13, wherein the illumination optical system has illumination light reflecting means for guiding the illumination light ejected by the illumination light ejection portion into the image pickup range of the image pickup means.

17. The endoscope system according to claim 13, wherein the illumination optical system has condensing means for narrowing an illumination range.

18. The endoscope system according to claim 16, wherein the illumination light reflecting means can change the optical path of the illumination light guided to the image pickup range of the image pickup means when the endoscope is separated.

19. The endoscope system according to claim 17, wherein the condensing means can change the illumination range of the illumination light.

20. The endoscope system according to claim 13, wherein the image pickup means has an objective optical system, polarizing means for changing at least two different luminous fluxes from the objective optical system in a different direction, an optical system for guiding the at least two different luminous fluxes to the same optical path, and polarized luminous flux transmitting means for alternately transmitting at least two luminous fluxes in the different polarized state, respectively.

21. An adapter applied to an endoscope system which comprises an endoscope having an insertion portion and having an illumination light incident portion for guiding illumination light to a base end of the insertion portion and an optical image ejection portion for ejecting a light from a subject guided through the insertion portion;
image pickup means having an image pickup device and provided with a first connection portion for attaching the endoscope so that the light from the subject ejected by the optical image ejection portion can be captured by the image pickup device, and
illumination light ejecting means having an illumination light ejection portion and provided with a second connection portion for attaching the endoscope so that the illumination light ejected by the illumination light ejection portion can enter the illumination light incident portion; and
wherein the adapter includes a first adapter connection portion capable of being attached to the first connection portion instead of the endoscope;
a second connection portion capable of being attached to the second connection portion instead of the endoscope;
an optical path for capturing an image of the subject by the image pickup device; and
an illumination optical system for guiding the illumination light ejected by the illumination light ejection portion into the image pickup region captured though the optical path.
